SEQUENCE LISTING (9 sequences)

SEQ ID NO: 1 - DNA, 20 bp, Artificial sequence
  Synthetic oligonucleotide
ccaacatmaggtgatgcgac

SEQ ID NO: 2 - DNA, 22 bp, Artificial sequence
  Synthetic oligonucleotide
tggctgtcccataamgcacmgt

SEQ ID NO: 3 - DNA, 24 bp, Artificial sequence
  Synthetic oligonucleotide
tggtatcctgtgtcccccagacct

SEQ ID NO: 4 - DNA, 19 bp, Artificial sequence
  Synthetic oligonucleotide
ggtccaagaggtggctggt

SEQ ID NO: 5 - DNA, 21 bp, Artificial sequence
  Synthetic oligonucleotide
ccgccttcctcttgagcataa

SEQ ID NO: 6 - DNA, 24 bp, Artificial sequence
  Synthetic oligonucleotide
ctcccactcttccaccttcgatgc

SEQ ID NO: 7 - DNA, 24 bp, Artificial sequence
  Synthetic oligonucleotide
cctctcttgctcagtgtccttgct

SEQ ID NO: 8 - DNA, 21 bp, Artificial sequence
  Synthetic oligonucleotide
cccaaccctaggccagccaag

SEQ ID NO: 9 - DNA, 23 bp, Artificial sequence
  Synthetic oligonucleotide
cacttgcagactccatttgccag

(19) United States
(12) Patent Application Publication
(10) Pub. No.: US 2006/0093580 A1
(43) Pub. Date: May 4, 2006

(54) Tolerogenic vaccine and method

(76) Inventors: Makio Iwashima, Martinez, GA (US); Nagendra Singh, Augusta, GA (US)

Correspondence Address: GREENLEE WINNER AND SULLIVAN P C, 4875 PEARL EAST CIRCLE, SUITE 200, BOULDER, CO, 80301, US

(21) Appl. No.: 11/267,040

(22) Filed: Nov. 4, 2005

Related U.S. Application Data
(60) Provisional application No. 60/625,403, filed on Nov. 4, 2004.

(51) Int. Cl. A61K 35/14; A61K 31/045

(52) U.S. Cl. 424/93.1; 514/724

(57) ABSTRACT

Methods and compositions are provided for treating autoimmune diseases such as diabetes, rheumatoid arthritis, inflammatory bowel disease, and other conditions involving undesired immune responses such as allergies, including food allergies, and graft-versus-host disease. In one embodiment disclosed, regulatory/suppressor T cells are selected or expanded in culture using a phospholipase D (PLD) inhibitor to prevent growth of effector T cells and a growth factor to stimulate the regulatory cells. Antigen-specific regulatory/regulatory T cells can be produced by this method. The regulatory T cells can then be administered to a patient in need of suppressive immunotherapy. In another embodiment, PLD inhibitor, growth factor, and an antigen for which antigen-specific suppressive immunotherapy is desired are administered to a patient via injection, oral or topical administration, or other means known to the art.

### CROSS-REFERENCE TO RELATED APPLICATIONS

This application claims priority to U.S. Provisional Application Ser. No. 60/625,403, filed Nov. 4, 2004, which is incorporated herein by reference to the extent not inconsistent herewith.

### STATEMENT AS TO FEDERALLY SPONSORED RESEARCH

This invention was made, at least in part, with Government funding, under NIH grant Nos. NIH 5RO1 AI047266, NIH 5KO2 AI049398, AI055022, and WB AR45212 and HL70046, and the Government therefore has certain rights in the invention.

### BACKGROUND OF THE INVENTION

Over the past decade peripheral lymphocytes, which include regulatory (also called “suppressor”) T cells, have been utilized in immunotherapy and gene therapy techniques for treating a number of human diseases.

U.S. Patent Publication No. 2002/0182730 (published Dec. 5, 2002 by M. L. Gruenberg, for “Autologous Immune Cell Therapy: Cell Compositions, Methods and Applications to the Treatment of Human Disease”) discloses an ex vivo method for expanding immune cells, including regulatory Th1 and Th2 cells that do not require exogenous IL-2. The expanded cell populations can be infused into patients for the treatment of autoimmune diseases. This method involves the use of various factors to enhance differentiation of regulatory T cells into Th1 or Th2 cells. U.S. Pat. No. 6,670,146 (issued Dec. 30, 2003 to Barrat et al. for “Regulatory T Cells; Methods,”) discloses a method for expanding regulatory T cells producing only IL-10 by contacting naive T cells derived from mouse spleen with an activator such as anti-CD3 along with a Vitamin D3/dexamethasone combination. No mention is made in these patent publications of CD4⁺CD25⁺ T cells.

CD4⁺CD25⁺ T cells are a recently-discovered subset of T cells which generally originate in the thymus. They can alternatively be generated, however, in the absence of an intact thymus. According to Karim et al., CD25⁺CD4⁺ regulatory T cells can be generated in the periphery from CD25⁻CD4⁺ precursors in a pathway distinct from that by which naturally occurring autoreactive CD25⁺CD4⁺ Treg cells develop (Karim et al. (2004), Alloantigen-induced CD25⁺CD4⁺ regulatory T cells can develop in vivo from CD25⁻CD4⁺ precursors in a thymus-independent process. J. Immunol. 172(2):923-928). Naturally present in the peripheral blood, these regulatory T lymphocytes are described as being small in number and capable of antigen-nonspecific suppression (Vigouroux, S. et al., Antigen-induced regulatory T cells, Blood. Jul. 1, 2004;104(1):26-33. Epub Mar. 16, 2004). In the absence of CD4⁺CD25⁺ T cells, the immune system can produce a stronger response to both self and foreign antigens. Elimination of these cells in mice leads to spontaneous development of various autoimmune diseases. (Takahashi, T., et al. (1998), “Immunologic self-tolerance maintained by CD4⁺CD25⁺ naturally anergic and suppressive T cells: induction of autoimmune disease by breaking their anergic/suppressive state,” International Immunology 10(12):1959-1980.) Like other T cells, the CD4⁺CD25⁺ subset are reported to demonstrate antigen specificity towards a diverse range of antigens. (Jiang, S., et al. (2003), “Induction of allopeptide-specific human CD4⁺CD25⁺ regulatory T cells ex vivo,” Blood 102(6):2180-2186). Without wishing to be bound by a particular theory, the inventors suppose that these CD4⁺CD25⁺ T cells may act to shut down an autoreactive effector T cell's function by shutting down that effector cell's ability to create or respond to IL-2, thus inhibiting the proliferation or function of that cell.

The CD4⁺CD25⁺ T cells have been found to be increased in mice tolerized to rheumatoid arthritis factor type II collagen (Min, So-Youn, et al. (2004), “Induction of IL-10 Producing CD4+CD25⁺ T cells in Animal Model of Collagen-Induced Arthritis by Oral Administration of Type II Collagen,” Arthritis Res. Ther. 6(3):R213-R219). Others report that co-injection of CD4⁺CD25⁺ T cells with CD4⁺ T cells protects recipient mice from inflammatory bowel disease (Banz, M. B., et al. (2004), “Suppression of CD4⁺ lymphocyte effector functions by CD4⁺CD25⁺ cells in vivo,” J. Immunol. 172(6):3391-3398). Furthermore, this T cell subset can inhibit bacterially-triggered intestinal inflammation (Maloy, K. J., et al. (2003), “CD4⁺CD25⁺ T(R) cells suppress innate immune pathology through cytokine-dependent mechanisms,” J. Exp. Med. 197(1):111-119).

These CD4⁺CD25⁺ T cells have been found to inhibit autoimmune diseases and tumor immunity, graft rejection, allergic disease, graft versus host disease, and acute and chronic infectious diseases. (Summary of Meeting, Regulatory/Suppressor T Cells, Mar. 10-15, 2004, Keystone Symposia, available online at the keystonesymposia website.

A large number of infectious diseases today are related to excessive or unregulated immune responses. U.S. Pat. No. 5,939,400 issued Aug. 17, 1999 to Steinman et al. for “DNA Vaccination for Induction of Suppressive T Cell Response,” discusses the role of pro-inflammatory CD4⁺ cells in inflammatory diseases caused by bacterial and viral infections including viral meningitis and bacterial meningitis, herpes encephalitis, and others. This patent provides a method of suppressing Th1 type T cell inflammatory response by vaccinating a patient with a DNA expression vector encoding the variable region of a T cell receptor to cause T cells expressing the variable region to produce Th2 cytokines to suppress the inflammatory T cell response. However, this vaccination method requires cumbersome cloning steps and knowledge of the variable region associated with the specific disease being treated.

U.S. Pat. No. 6,464,978 issued Oct. 15, 2002 to Brostoff et al. for “Vaccination and Methods Against Multiple Sclerosis Resulting from Pathogenic Responses by Specific T Cell Populations,” discusses the use of a vaccine composed of a T cell receptor (TCR) or a fragment thereof corresponding to a TCR present on the surface of autoaggressive T cells responsible for various autoimmune pathologies. This method, however, requires isolation of the relevant T cells and identification of appropriate TCRs or fragments.

Antigen receptor stimulation activates phospholipase D (PLD) in lymphocytes (Stewart, S. J. et al. (1991), Cell Regul. 2:841-850; Reid, P. A. et al., Immunology (1997), 90:250-256 (February 1997); Gilbert, J. J. et al. (1998), J Immunol 161:6575-6584; Gruchalla, R. S. et al. (1990), J Immunol 144:2334-2342). Activated PLD catalyses the hydrolysis of phosphatidylcholine (PC) to phosphatidic acid (PA) and choline (Exton, J. H. (2002), Rev Physiol. Biochem. Pharmacol. 144:1-94). PLD has been shown to play a role in events triggered by the receptors that are coupled to the immunoreceptor tyrosine-based activation motif (ITAM) (e.g. Fcy receptor-mediated phagocytosis, degranulation, exocytosis, membrane ruffling) (Melendez, A. J. (2002), Semin. Immunol. 14:49-55; Chahdi, A., et al. (2002), Mol. Immunol. 38:1269-1276; Cockcroft, S. et al. (2002), Mol. Immunol. 38:1277-1282). Inhibitors of Phospholipase D are discussed in Exton (2002), J. H., “Phospholipase D—Structure, Regulation and Function, Reviews of Physiology, Biochemistry, and Pharmacology 44:1-94. Adenosine has been described as inhibiting PLD activation in neutrophils (Thibault, N., et al. (2000), Blood 95(2):419-527; Grenier, S. et al. (2003), J. Leukoc. Biol. 73(4):530-539).

Although previous studies show that TCR engagement can induce PLD activity in T cells, the biological significance of this for immune responses is unknown. In the presence of primary alcohols such as 1-butanol, PLD favors catalysis of transphosphatidylation over hydrolysis and produces phosphatidylalcohol (Exton, J. H. (2002), Rev. Physiol. Biochem. Pharmacol. 144:1-94). As a result, production of phosphatidic acid (PA) is significantly reduced and PA-derived diacylglycerol (DAG) production is also decreased because phosphatidylalcohols are poorly metabolized.

U.S. Patent Publication 2004/0029244, published Feb. 12, 2004, by Williger, for “Phospholipase D Effectors for Therapy and Screening” discloses that phospholipase D inhibitors, in particular primary alcohols such as 1-butanol, are useful as drugs for the treatment of disorders wherein matrix metalloproteinase enzyme expression levels are pathological. When enzyme levels are suppressed, growth of abnormally proliferating cells such as cancer cells forming tumors and metastatic lesions is disabled. This patent publication does not teach or suggest the use of phospholipase D inhibitors for preferential selection or expansion of regulatory T cells.

There is a need for efficient in vitro and in vivo production and selection or expansion of these regulatory/suppressor T cells for treatment of autoimmune disorders, including effective immune suppression during organ transplantation, as well as other diseases. There is also a need for a simple, efficient procedure that allows for specific suppression of immune responses.

All publications referred to herein are incorporated herein by reference to the extent not inconsistent with the teachings hereof.

### SUMMARY

This invention provides a method for selectively increasing proliferation of regulatory T cells compared to effector T cells comprising: contacting a T cell population, wherein the population comprises regulatory T cells and optionally effector T cells with a phospholipase D (PLD) inhibitor in an amount effective to selectively inhibit said effector T cells; activating the regulatory and effector T cells, and allowing proliferation of the regulatory T cells and/or elimination of the effector T cells.

Optionally, the T cell population is contacted with a growth factor in an amount sufficient to promote proliferation of the regulatory T cells.

The method can be performed in vitro, preferably for the purpose of growing up clinically relevant numbers of regulatory T cells for use in adoptive immunotherapy to suppress immune responses, or can be performed in vivo, by means of vaccination or other form of administration to a patient in need of immunosuppression, of PLD inhibitor, optionally, a growth factor, and optionally an activating antigen.

The regulatory T cells can be effective to suppress effector T cells in general, or can be “antigen specific,” i.e., activated by a specific antigen so as to be effective to suppress effector T cells which respond only to that specific antigen.

The methods of this invention are useful for treating autoimmune diseases such as rheumatoid arthritis, lupus, multiple sclerosis, inflammatory bowel disease, insulin-dependent diabetes mellitus, autoimmune thyroid disease, anti-tubular basement membrane disease (kidney), Sjogren's syndrome, ankylosing spondylitis, uroetinitis, and undesirable immune reactions such as allograft rejection, transplant rejection, allergies including food allergies, immune responses initiated by damage to immunologically-privileged sites such as brain and eyes, e.g., by infection, stroke, and asthma. Preferably, treatment is begun before symptoms arise, and the patient treated is one at risk of developing such undesirable immune reactions.

In embodiments of this invention, compositions of matter suitable for administration to patients in need of immunosuppression, including antigen-specific immunosuppression are also provided comprising clinically relevant numbers of regulatory T cells, which can be antigen-specific regulatory T cells. Such compositions can be administered in pharmaceutically suitable carriers.

In other embodiments of this invention, compositions of matter suitable for administration to patients in need of immunosuppression comprise a PLD inhibitor, optionally, a growth factor such as IL-2, and optionally, an activating antigen for which antigen-specific immunosuppression is desired.

### BRIEF DESCRIPTION OF THE FIGURES

FIG. 1 Inhibition of phospholipase D signaling leads to induction of suppressive activity by CD4 T cells.

(A) Effect of PLD inhibition on anti-CD3-induced cell division. Splenic CD4 and CDB T cells were labeled with CFSE and stimulated with anti-CD3 and γ-irradiated antigen-presenting cells (APCs). 1-butanol or t-butanol was added to the culture as shown above each panel.

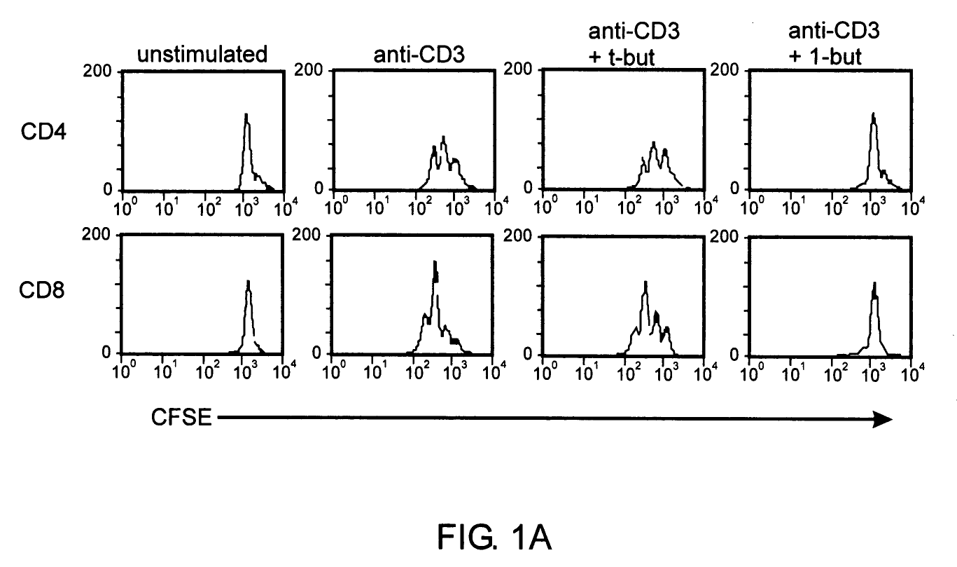

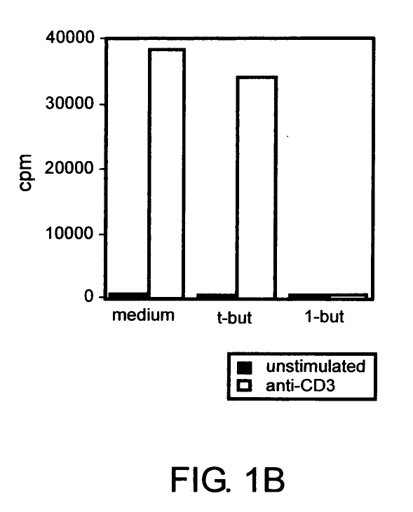

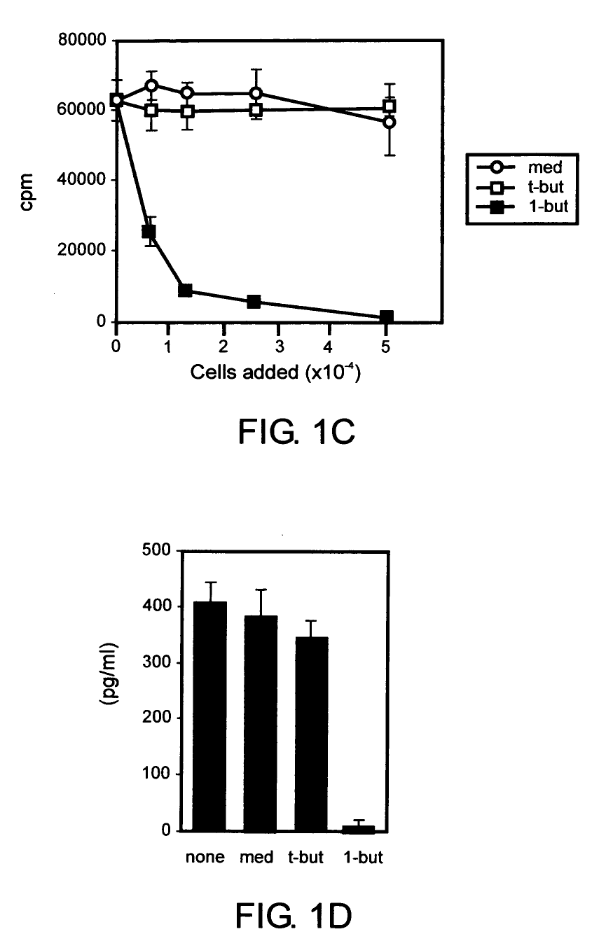

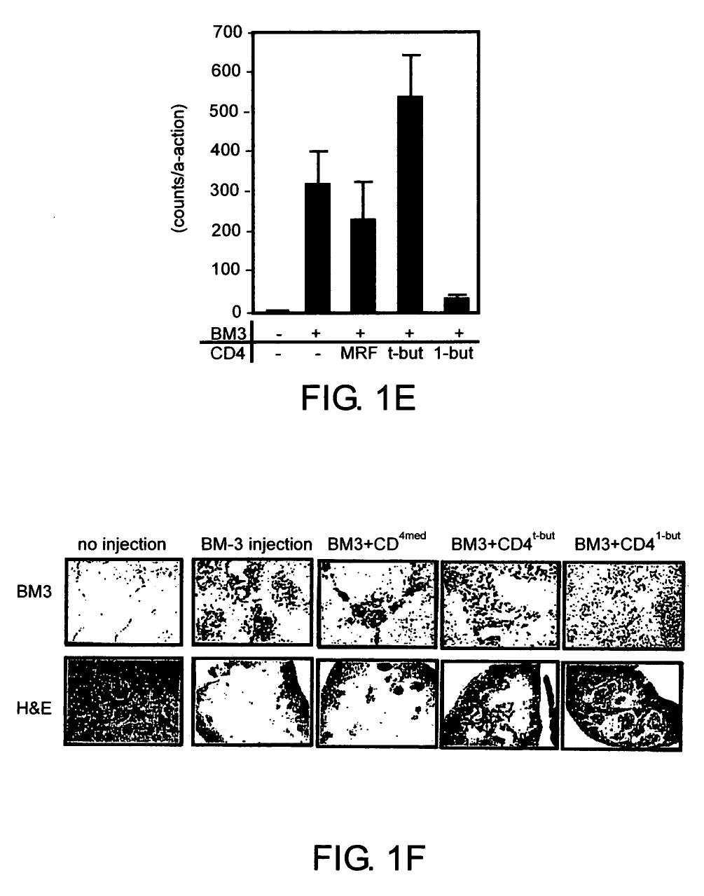

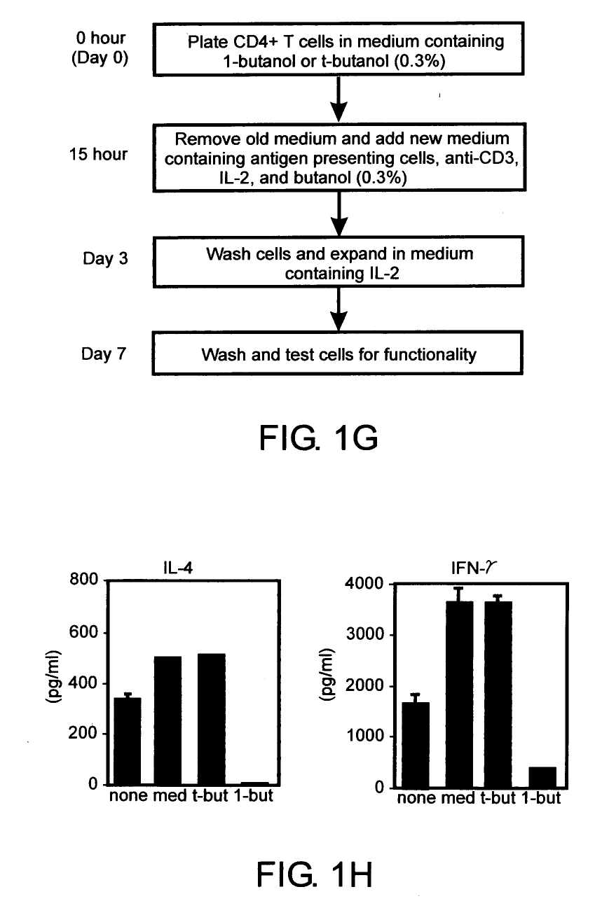

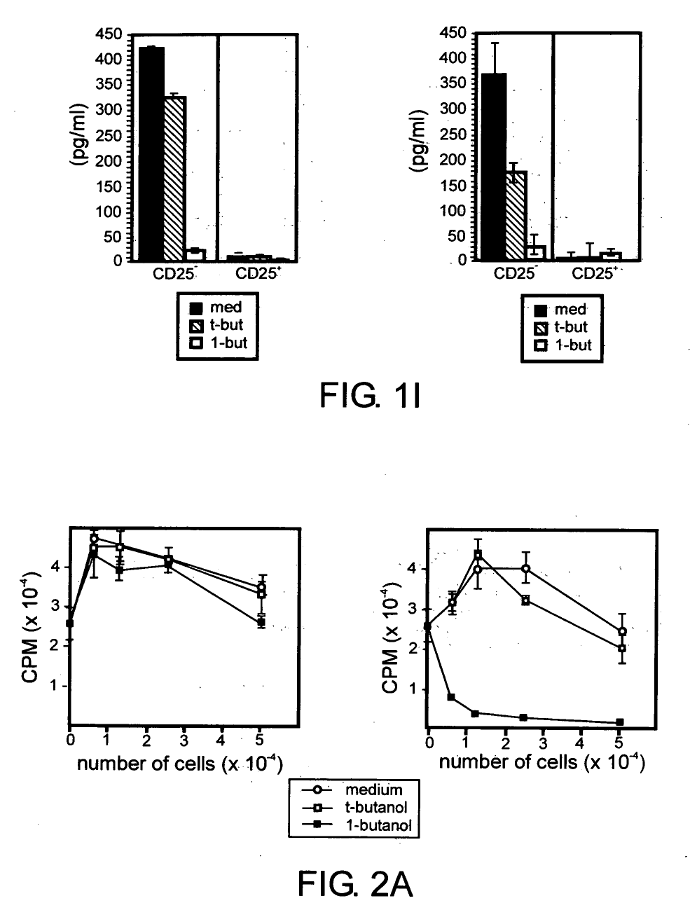

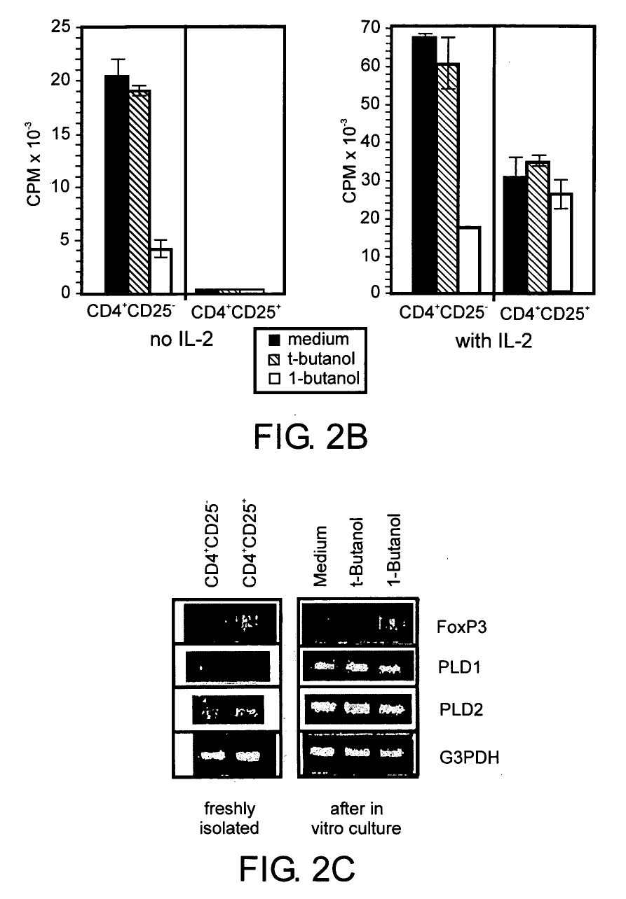

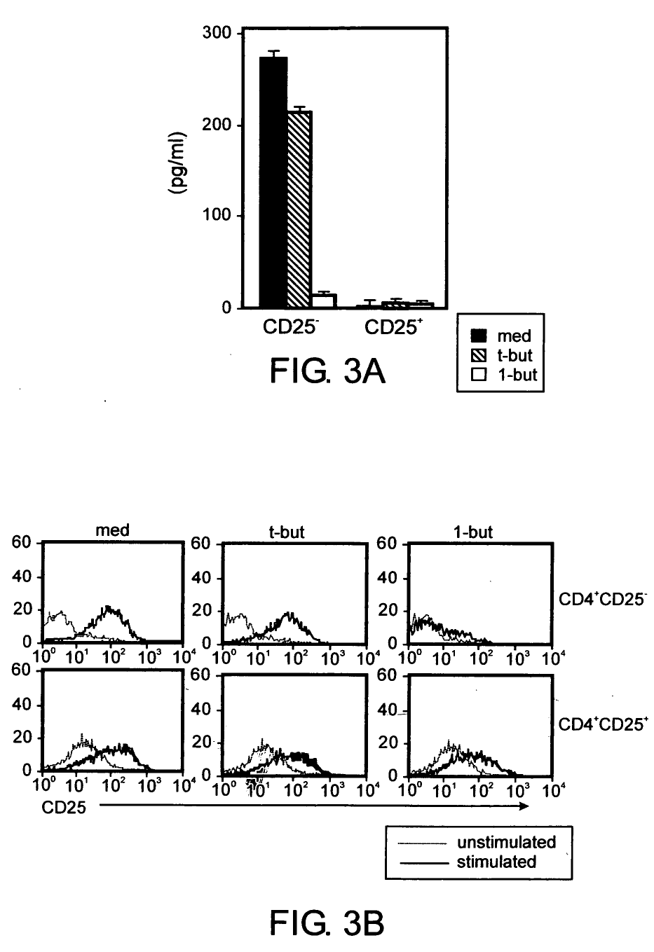

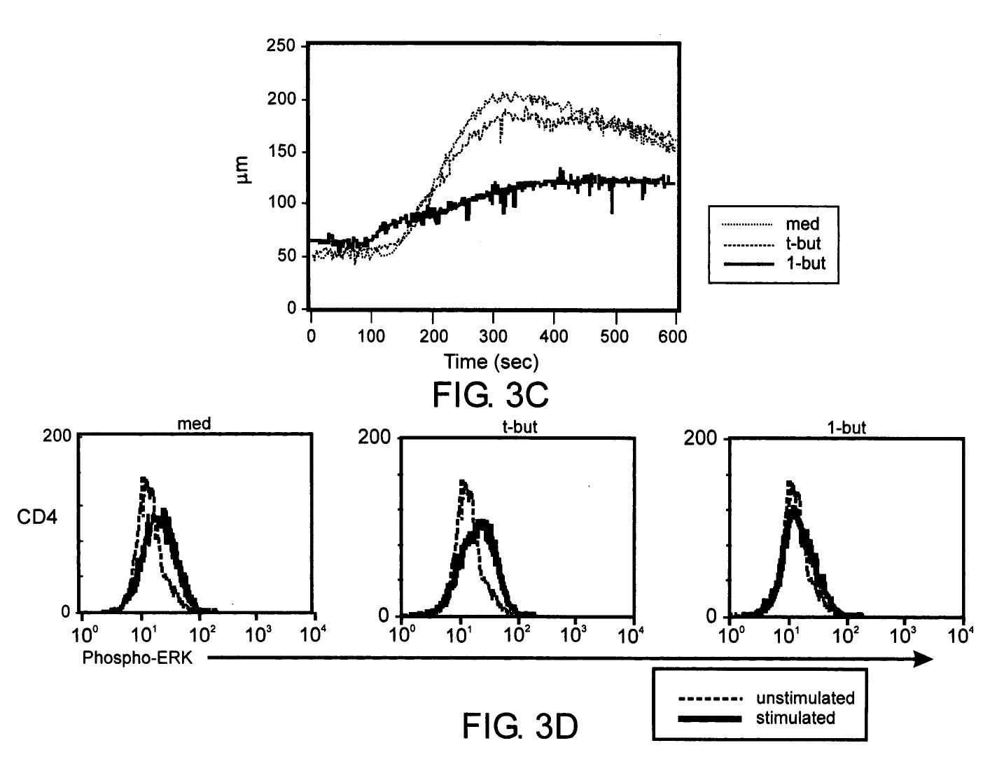

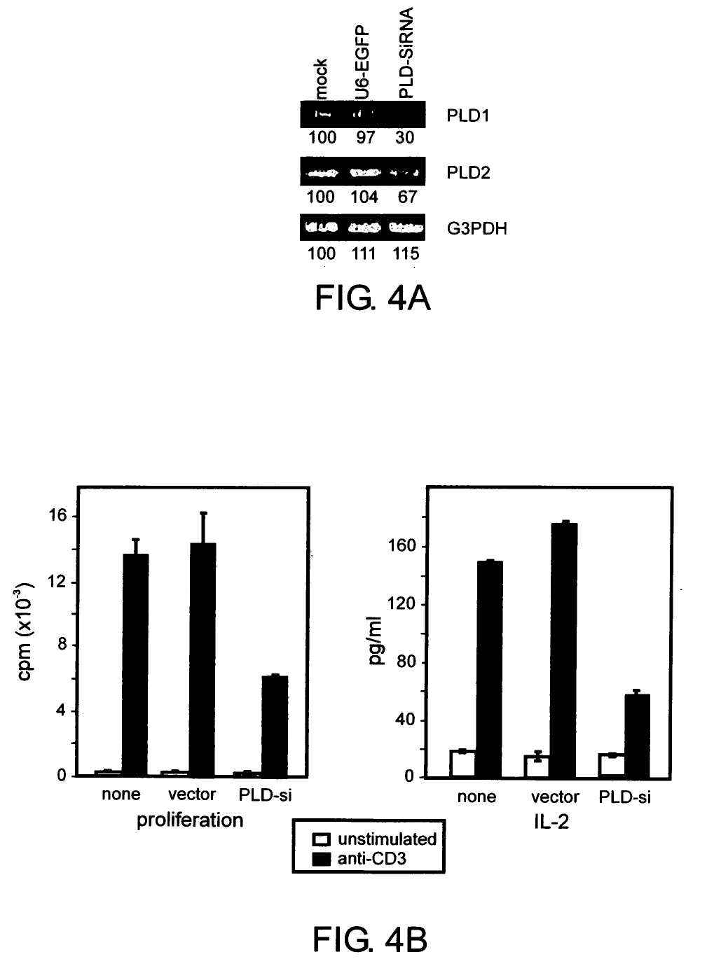

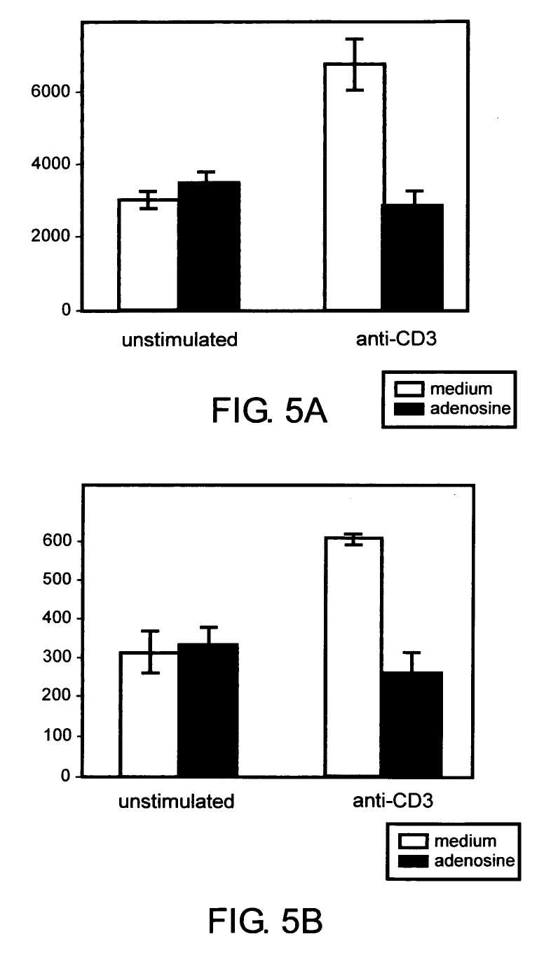

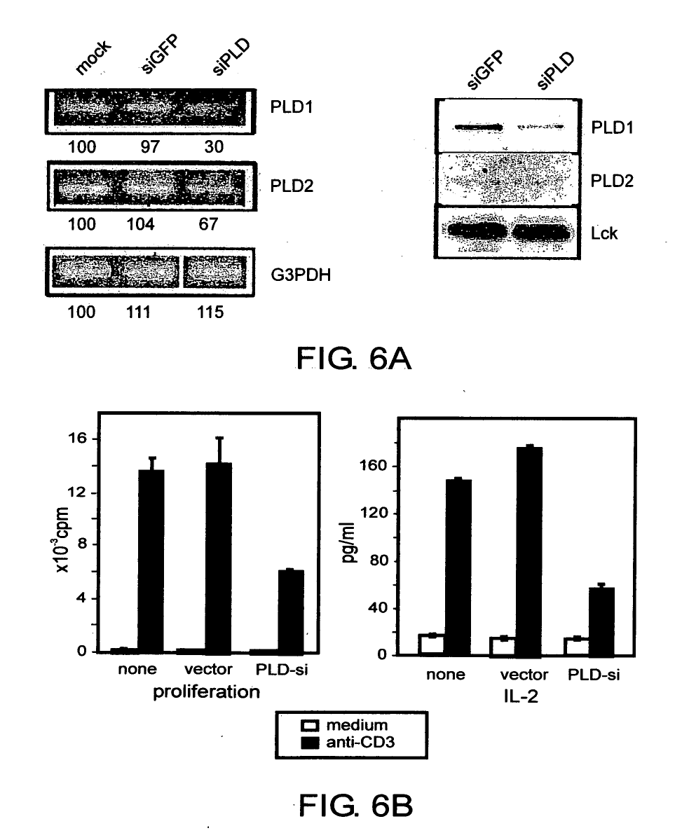

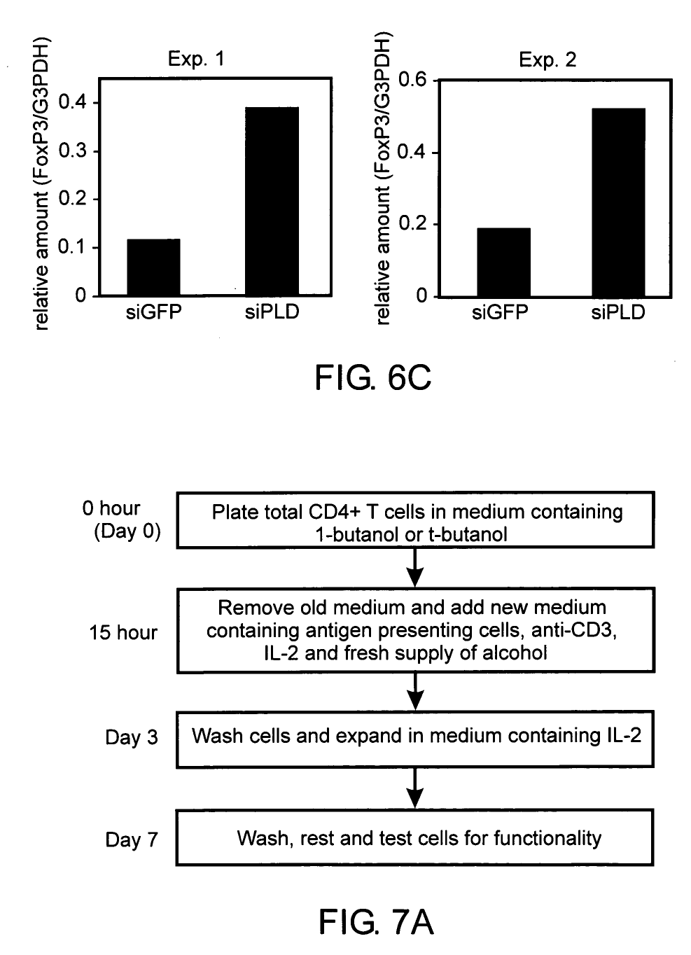

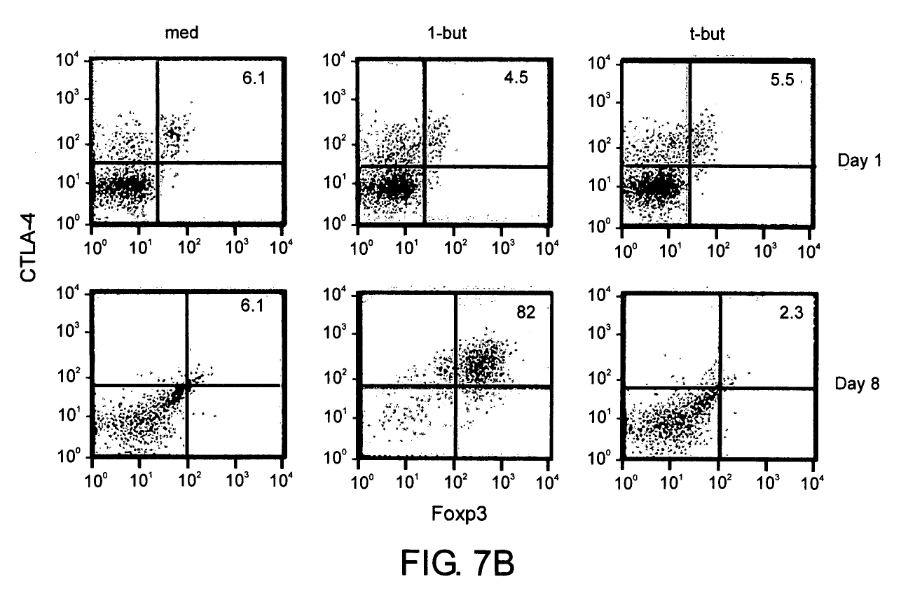

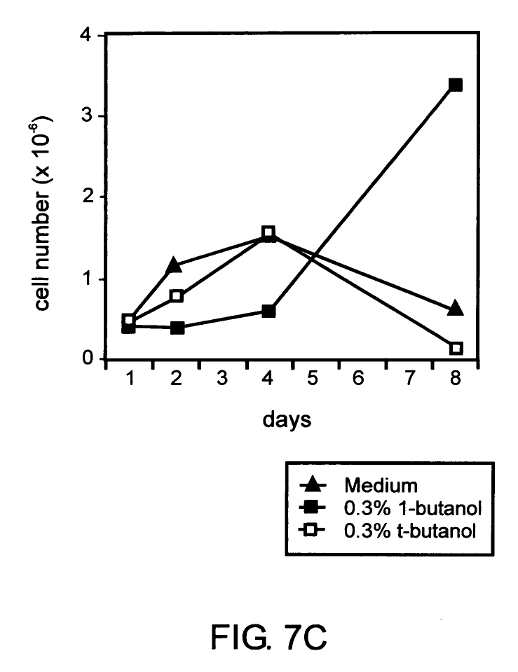

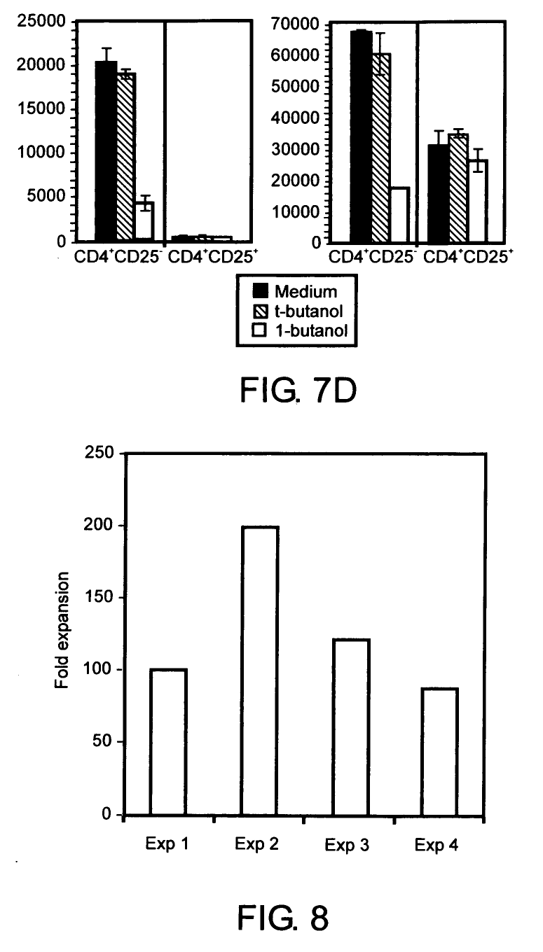

(B) Secondary response of CD4^{1-but} CD4^{t-but} and CD4^{med} cells to anti-CD3 stimulation. Cells were cultured with t-butanol, 1-butanol, or medium alone as shown in FIG. 1G, and were restimulated with anti-CD3 plus APCs. ³H-thymidine uptake was measured on day 3. Means of triplicate data from a representative experiment are shown.

(C) Suppressive effect of CD4^{1-but} cells on anti-CD3-induced T cell proliferation. CD4⁺CD25⁻ T cells (2.5×10⁴ cells) were cultured with graded doses of CD4^{med} cells (open circles), CD4^{t-but} cells (open squares) or CD4^{1-but} cells (closed squares) for 72 hours with anti-CD3 antibody and APCs. Proliferation of cells was measured as in (B).

(D) Suppressive effect of CD4^{1-but} cells on anti-CD3-induced IL-2 production. Cells were cultured as in (C) using equal numbers of CD4⁺CD25⁻ cells and CD4^{med} cells, CD4^{1-but} cells, or CD4^{t-but} cells. Production of IL-2 after 24 hours of co-culture was measured.

(E) In vivo function of CD₄^{1-but} cells. F1 mice were injected intravenously with syngenic CD4^{med} cells, CD₄t-but cells, or CD4^{1-but} cells. 24 hours later, all mice were injected with BM3 splenocytes. Four days after the second injection, mice were sacrificed and spleens were examined for follicular architecture and presence of BM3 TCR positive T cells using a monoclonal antibody (Ti98) specific to BM3 TCR. Transgenic T cells were counted by image analysis software (BioQuant). Average numbers of three stained areas are shown for each sample.

(F) Expansion and tissue destruction by BM3 T cells. Representative images from mice described in (E) are shown. Upper panels show immunohistochemical staining of the tissues with anti-BM3 idiotype antibody (×20). Dense red staining shows BM3 cells injected and proliferated in the spleens. Lower panels show hematoxylin and eosin (H&E) staining (×4).

(G) Flow chart for the procedure used to produce CD4^{1-but}, CD4^{t-but}, and CD4^{med} cells. Purified CD4 T cells were pre-incubated with 1-but (0.3%), t-but (0.3%), or medium alone for 15 hours. There was no difference among the three groups in terms of viability or surface antigen expression after pre-incubation (not shown). These cells were then stimulated with anti-CD3 antibody and γ-irradiated APCs and exogenous IL-2. 1-butanol or t-butanol was added to give a final concentration of 0.3%. On day 3, cells were washed and plated in medium containing IL-2 but no anti-CD3 or alcohol. On day 7, cells were washed and used for the functional analysis.

(H) Effect of CD4^{med}, CD4^{t-but}, and CD4^{1-but} cells on cytokine production by CD4⁺25⁻ T cells. CD4⁺25⁻ T cells were stimulated and co-cultured with CD4^{med} cells (med), CD4^{t-but} cells (t-but), or CD4^{1-but} cells (1-but) as described in FIG. 1D. IL-4 and IFN-γ in the culture supernatants were measured by ELISA.

(I) Cytokine production by 1-but and t-but-treated CD4⁺25⁻ and CD4⁺25⁺ T cells. CD4⁺25⁻ and CD4⁺25⁺ T cells were stimulated as described in FIG. 3A. The same supernatants were used to measure the amount of IL-4 (left panel) and IFN-γ (right panel). Closed bars show medium-treated T cells, gray bars show t-but- treated T cells, and open bars show 1-but-treated T cells.

FIG. 2 Preferential expansion of CD4⁺CD2S⁺ T cells in the presence of 1-butanol.

(A) Suppressive activity of CD4^{med} cells (open circles), CD4^{t-but} cells (open squares) or CD4^{1-but} cells (filled squares) prepared from either CD4⁺CD25⁻ (left panel) or total CD4⁺ (right panel) cells. Suppression of freshly isolated CD4⁺CD25⁻ cells was measured as in FIG. 1C. 1×10⁶ (total CD4⁺ or CD4⁺CD25⁻) cells were treated with 1-butanol and 3.6×10⁶ and 5×10⁵ cells were recovered, respectively, indicating a majority of CD4^{1-but} cells are derived from CD4⁺CD25⁺ T cells.

(B) 1-butanol effect on the proliferative response of CD4⁺CD25⁻ and CD4⁺CD25⁺ T cells to anti-CD3 or anti-CD3⁺ exogenous IL-2. 1-butanol (open bars), t-butanol (gray bars), or medium (black bars) was added to the culture at the beginning of stimulation. Proliferation of cells was measured as in FIG. 1B.

(C) Expression of mRNA encoding Foxp3, PLD1 and PLD2 by CD4 T cells subpopulations. mRNA levels for genes indicated were determined by semi-quantitative RT-PCR of freshly isolated CD4⁺CD25⁻, CD4⁺CD25⁺ (left panel), and CD4^{med}, CD4^{t-but} or CD4^{1-but} cells (right panel). Quality and quantity of mRNA was confirmed to be equivalent by glyceraldehydes-3-phosphate dehydrogenase (G3PDH) mRNA level as shown in the bottom panels.

FIG. 3 Effect of PLD signal inhibition on activation-induced events:

(A) Effect of 1-butanol on anti-CD3-induced IL-2 production. CD4⁺CD25⁻ and CD4⁺CD25⁺ T cells were stimulated with anti-CD3 in the presence of medium alone (black bars), t-butanol (gray bars), or 1-butanol (open bars). IL-2 production was determined by ELISA of culture supernatants obtained after 24 hours of stimulation.

(B) 1-butanol effect on expression of CD25 by CD4⁺CD25⁻ and CD4⁺CD25⁺ T cells. CD4⁺CD25⁻ and CD4⁺CD25⁺ T cells were treated with 1-butanol, t-butanol, or with medium alone and were activated with anti-CD3 and APCs as described in FIG. 1. After 16 hours of stimulation, expression of CD25 was analyzed by flow cytometry. Dotted lines represent the staining level of unstimulated cells.

(C) 1-butanol effect on anti-CD3-induced elevation of intracellular Ca2⁺. CD4 T cells were labeled with Fura2-AM and activated with biotin conjugated anti-CD3 and streptavidin. 1-butanol (dark line), t-butanol (thin line) or medium (dotted line) was added together with stimulating antibody. Levels of Ca2⁺ were determined by the ratio of fluorescence at 340/380 nm.

(D) Effect of 1-butanol on antl-CD3-induced ERK activation. CD4 T cells were stimulated with anti-CD3 and APCs for 3 hours and stained with antibody against phospho-ERK. Stimulation was carried out in the presence of 1-butanol, t-butanol, or medium alone as shown above each panel. Thick lines show the data from stimulated cells and thin lines show data from unstimulated cells.

FIG. 4 Functional effects of PLD gene knock-down:

(A) Effect of siRNA on PLD mRNA expression. Purified CD4 T cells were transfected with the expression cassette targeted toward both PLD1 and 2. As controls, cells transfected with the expression cassette for EGFP (U6-EGFP) or with no DNA (mock) were examined. 18 hours after transfection, total RNA was harvested and mRNA levels for PLD1, PLD2 and G3PDH were determined by semi-quantitative RT-PCR. The levels of PLD1 and PLD2 mRNA in each transfectant were compared against mock transfectants by densitometry and their relative percentages are shown below each lane. The efficiency of each transfectlon was comparable, as determined by co-transfecting a Renilla-luciferase expression construct (not shown).

(B) Effect of PLD siRNA on anti-CD3-induced T cell proliferation and IL-2 production. CD4 T cells transfected as described in (A) were stimulated with anti-CD3 and APCs. Proliferation (after 72 hours) and IL-2 production (after 24 hours) were measured for each sample.

FIG. 5 Effect of adenosine on TCR-induced PLD activation. (A) Phosphatidic acid production by primary CD4 T cells stimulated with anti-CD3 antibodies in the presence of ethanol (open bar) and adenosine (closed bar) compared with unstimulated T cells. (B) Phosphatidylethanol (Pet) production by primary CD4 Mouse CD4 T cells stimulated with anti-CD3 antibodies in the presence of ethanol (open bar) and adenosine (closed bar) compared with unstimulated T cells.

FIG. 6 Effect of PLD gene knockdown on T cell activation. (A) Effect of siRNA on PLD expression. (B) Effect of PLD siRNA on anti-CD3-induced T cell proliferation and IL-2 production. (C) Foxp3 expression by cells treated with siRNA for PLD.

FIG. 7 Cells expanded in in vitro culture with 1-butanol are enriched for Foxp3 positive cells.

(A) A schematic presentation of the procedure utilized in these experiments to expand T cells in the presence of 1 alcohol.

(B) Foxp3 and CTLA4 levels were determined by flow cytometry analysis of CD4^{med}, CD4^{t-but} or CD4^{1-but} cells at day 1 (upper panels) and day 8 (lower panels). Both 1-butanol and t-butanol were added to the medium at final concentration of 0.3%.

(C) Absolute cell numbers of Foxp3 positive cells during the culture of CD4 T cells with 1-butanol (closed square), t-butanol (open square), and medium alone (closed triangle).

(D) CD4⁺CD25⁻ and CD4⁺CD25⁺ T cells (2×10⁴ cells/well) were stimulated by anti-CD3 in the absence (left panel) or presence (right panel) of exogenous IL-2. 0.3% 1-butanol (open bars), 0.3% t-butanol (gray bars), or medium (black bars) was added to the cultures at the beginning of stimulation. [3H]-thymidine uptake was measured 72 hours later.

FIG. 8 Plate-bound antibody-based stimulation of CD4⁺CD25⁺ cells.

### DETAILED DESCRIPTION

“Regulatory T cells” as used herein are CD4⁺CD25⁺ T cells and can alternatively be referred to as suppressor T cells.

“Effector T cells” as used herein includes all T cells whose activities are suppressed by the function of the regulatory T cells, including CD4⁺CD25⁻ T cells, CD8 T cells, and Th1 and Th2 helper T cells, γδ T cells, and subsets thereof.

“Increasing proliferation of cells” means to measurably increase the number of cells present. In an embodiment, the increasing can be relative to a proportion of a subset of the cells, e.g., increasing the number of regulatory T cells relative to the number of effector T cells.

“Contacting” the cells with a phospholipase D (PLD) inhibitor or a growth factor can be done in vivo or in vitro by any means known to the art. When the method is practiced in vivo the growth factor can be one that is endogenously generated in situ when an activating antigen is administered to the patient, or the growth factor can be administered to the patient along with the PLD inhibitor and activating antigen.

Phospholipase D inhibitors are known to the art. See, e.g., U.S. Patent Publication 2004/0029244 and Exton (2002), J. H., “Phospholipase D—Structure, Regulation and Function, Reviews of Physiology, Biochemistry, and Pharmacology 44:1-94, incorporated herein by reference. They include compounds having at least one primary hydroxyl or at least one primary sulfhydryl group conjugated to a physiologically acceptable chemical moiety through a linear spacer group n carbon atoms or n heteroatoms in length wherein n is an integer from 3 to 20. Preferred compounds are selected from the group consisting of 1-propanol, 1-butanol, ethanol, 1-propanthiol, 1-butanthiol and mixtures thereof. The physiologically acceptable chemical moiety is any atom or chemical group which serves to enhance the efficacy of the conjugated PLD inhibitor, e.g., through enhancing chemical or physiological stability, permeability, affinity, solubility, or biological efficacy of the PLD inhibitor. It can also serve as a reporter group by incorporating a radioactive or other detectable group. Examples of physiologically acceptable conjugated moieties are atoms or chemical groups selected from the group consisting of hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl, arylalkyloxylcarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, saccharides, polysaccharides, glycosaminoglycans, salicylates, steroids, hydroxysteroids, purines, pyramidines, nucleosides, amino acids, peptides, glycerides, poly-glycerides, glycols, polyglycols, lipids, individual isomers and combinations thereof.

Other PLD inhibitors include some compounds which are also inhibitors of serine proteases. A serine protease is a hydrolytic enzyme which has a serine residue at its active site and cleaves peptides or proteins. In some cases, serine proteases also cleave esters. An example of a serine protease inhibitor which is also an inhibitor of PLD is the compound 4-(2-aminoethyl)-benzenesulfonyl fluoride. This compound is a polar compound and of low permeability to biological membranes, such as lipid bilayers, cell membranes, mucosa, gastrointestinal lining, kidney, tubules, or blood-brain barrier. According to the invention, PLD inhibitors are conjugated to a physiologically acceptable moiety to enhance the chemical stability of the inhibitor, physiological stability of the inhibitor, cell membrane permeability of the inhibitor, or a combination of these. According to the invention, conjugating a serine protease inhibitor which is also a PLD inhibitor to a physiologically acceptable moiety has the advantage of achieving a greater inhibition of intracellular PLD activity, wherein the conjugated moiety is a lipophilic or essentially hydrophobic group which enhances the permeability of the inhibitor moiety to a biological membrane, such as a lipid bilayer, a cell membrane, a mucosa layer, the gastrointestinal mucosa, the kidney tubule, the blood-brain barrier, or a combination thereof.

Adenosine and its derivatives can be used as PLD inhibitors in the methods and compositions of this invention. As used herein, an adenosine derivative is a compound in which additional pendent NH₂ moieties may be present on the purine ring, and on the NH₂ moiety(ies), and/or to substitute for both hydrogens thereof; or one or more ring-pendant riboside hydroxyls are replaced with H, methyl, ethyl, propyl, butyl, or other C1-C4 groups including C1-C4 alcohols, carbonyls and acids, amine, amine substituted with the same or phenyl or substituted phenyl rings; or the foregoing groups may be present at the 2′ or 3′ positions.

Preferably the PLD inhibitor is an inhibitor of the PLD isoform PLD1. CD4⁺CD25⁺ regulatory T cells do not require this isoform for proliferation; however, CD4⁺CD25⁻ T cells do require this isoform to proliferate.

An effective amount of PLD inhibitor to inhibit growth and proliferation of effector T cells can be readily determined by one skilled in the art without undue experimentation, and is generally an amount which will result in contact of the cells with a solution containing less than about 1% of the inhibitor, more preferably about 0.3 to about 0.5% of the inhibitor, and most preferably about 0.3%, Thus in vitro a culture medium comprising the PLD inhibitor in the foregoing amounts would be an effective amount. In vivo, the amount of PLD inhibitor to be administered will depend on clinical considerations such as the size and weight of the patient, and whether or not the administration is to be local or systemic.

An effective amount of PLD inhibitor to inhibit growth of effector T cells is an amount sufficient to measurably inhibit the growth of these cells, preferably an amount which will inhibit the growth of effector T cells such that the ratio of effector to regulatory T cells after treatment with the PLD inhibitor is about 1:4 or less, and preferably about 1:9 or less.

Suitable growth factors with which the T cells can be contacted to promote proliferation of the regulatory T cells are selected from the group consisting of IL-7, TGF-β, IL-12, IL-10, and IL-2, preferably IL-2.

The amount of growth factor which is effective to promote proliferation can be readily determined by one skilled in the art without undue experimentation, and is generally an amount which will result in contact of the cells with a solution containing about 10 up to about 100 units/ml of the growth factor, more preferably about 30 to about 60 units/ml of the growth factor, and most preferably about 50 units/ml of the growth factor. Thus in vitro a culture medium comprising the growth factor in the foregoing amounts would be an effective amount. In vivo, administration of growth factor may not be necessary, depending on whether administration of the activating antigen causes endogenous production of sufficient growth factor or not. If it is necessary, the amount of growth factor to be administered will depend on clinical considerations such as the size and weight of the patient, relative health, and whether or not the administration is to be local or systemic. Care should be taken not to administer so much growth factor that cytokine release syndrome occurs.

An effective amount of growth factor to promote growth of the regulatory T cells is an amount sufficient to measurably promote proliferation of these cells, preferably an amount which will promote the growth of regulatory T cells such that the ratio of regulatory to effector T cells after treatment with the PLD inhibitor and growth factor is about 4:1 or more and preferably about 9:1 or more.

Activation of the T cells can be done by contacting them with an antigen to which they react, such as anti-CD3 antibody, or other such antigens known to the art to which all T cells react, or with a specific antigen such as an allergen, allogenic major histocompatibility complex classes (MHCs), proteins from immunological privileged sites, self antigens that are associated autoimmune diseases, or viral and bacterial antigens that initiate neuronal damages by immune responses. An effective amount of antigen to activate the T cells can be readily determined by one skilled in the art without undue experimentation, and is generally an amount which will result in contact of the cells with a solution containing about 0.01 mg to about 1 mg/ml of protein antigen or about 1 to about 100 μg/ml peptide antigen, more preferably about 0.1 to about 1 mg/ml of protein antigen or about 10 to about 100 μg/ml peptide antigen, and most preferably about 0.2 mg/ml protein or about 0.2 μg/ml peptide, Thus in vitro a culture medium comprising the antigen in the foregoing amounts would be an effective amount. In vivo, the amount of antigen to be administered will depend on clinical considerations such as the size and weight of the patient, and whether or not the administration is to be local or systemic.

An effective amount of antigen to activate the T cells is an amount sufficient to measurably cause proliferation of the regulatory T cells, preferably to clinically relevant numbers.

“Clinically-relevant numbers” with respect to in vitro embodiments of this invention preferably means an amount suitable for effective adoptive immunotherapy involving administration of preferably autologous suppressive T cells to a patient in need of such therapy, i.e., therapeutically effective numbers such as greater than 10⁸ and more preferably greater than 10⁹. In vivo embodiments should produce at least such numbers of regulatory T cells, and preferably more. A clinically-relevant number of cells is a therapeutically effective number that is at least sufficient to achieve a desired therapeutic effect.

“Allowing proliferation” of the regulatory T cells means to permit a period of time sufficient for therapeutically effective numbers of the regulatory T cells to be produced in vivo or in vitro. Preferably a ratio of regulatory T cells to effector T cells of about 1:2, or more preferably about 1:1 is used to reduce the number of effector T cells.

Collecting and culturing the cells can be done by any means known to the art, e.g., those disclosed in U.S. Patent Publication 2002/0182730, incorporated herein by reference to the extent not inconsistent herewith.

Methods of this invention can be performed in vitro, preferably for the purpose of growing up clinically relevant numbers of regulatory T cells for use in adoptive immunotherapy to suppress immune responses. Adoptive immunotherapy involves administering suppressive T cells to a patient in need of immunosuppression. Autologous cell therapy is a form of adoptive immunotherapy in which a patient's own cells are used in the method for proliferating suppressive T cells and then the proliferated (also called “expanded”) suppressive T cells are administered back to the patient. These adoptive immunotherapy methods can be used for general immunosuppression or antigen-specific immunosuppression, and comprise: collecting T cells from a donor, who in the case of autologous cell therapy, will be the patient in need of the suppressive immunotherapy; activating said T cells by contacting them with an antigen, and when immunosuppression of reaction to a specific selected antigen is desired, the antigen used is a selected specific antigen; culturing said T cells in the presence of a PDL inhibitor such as 1-butanol or 1-propanol and a growth factor such as IL-2, in an effective amount to promote proliferation of suppressive T cells in culture; expanding the suppressive T cells in said culture until a clinically relevant number of regulatory T cells capable of suppressing the immune response; and administering said regulatory T cells to a patient in need of said immunosuppression. An activating antigen, which can be a specific antigen for which immunosuppression is desired, can be co-administered with the suppressive T cells and/or PLD inhibitors.

Therapeutically-effective compositions of this invention comprising clinically-relevant numbers of regulatory T cells can be administered by any means known to the art, e.g., orally, nasally, ocularly, topically, rectally, or parentally in a unit dosage injectable form (solution, suspension, emulsion) in association with a pharmaceutically acceptable parental vehicle. Such vehicles are inherently nontoxic and nontherapeutic. The regulatory T cells can be administered in aqueous vehicles such as a saline solution, or buffered vehicles with or without various additives and/or diluting agents. They will normally be administered intravenously, though it is possible to administer them subcutaneously, intradermally, or intramuscularly by injection. The proportion of therapeutic entity and additive can be varied over a broad range so long as all are present in effective amounts. The therapeutic composition is preferably formulated in purified form substantially free of aggregates, other proteins, endotoxins, and the like, at concentrations of about 1 to 30×10⁷ cells/ml, preferably about 1 to 10×10⁷ cells/ml. Preferably, the endotoxin levels are less than 2.5 EU/ml. See, e.g., Avis, et al. (eds. 1993) Pharmaceutical Dosage Forms: Parenteral Medications 2d ed., Dekker, N.Y.; Lieberman, et al. (eds. 1990) Pharmaceutical Dosage Forms: Disperse Systems, Dekker, N.Y.; Fodor, et al. (1991) Science 251:767-773; Coligan (ed.) Current Protocols in Immunology; Hood, et al., Immunology Benjamin/Cummings; Paul (ed. 1997) Fundamental Immunology 4^{th} ed., Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

The compositions of this invention can be administered in pharmaceutical carriers known to the art for administering pharmaceuticals via the foregoing routes, including tablets, pellets for implantation, inhalation sprays and infusions, eye drops, intravenous, intramuscular, and subcutaneous injection carriers, and creams and ointments and other topical carriers. Preferably, the carrier includes a delivery vehicle allowing slow release of the antigen and PLD inhibitor, for example microbeads capable of absorbing these components. Administration of the components preferably takes place over a period of about one month.

Preferably, an administration regimen maximizes the amount of therapeutic composition delivered to the patient consistent with an acceptable level of side effects. Accordingly, the amount of therapeutic composition delivered depends in part on the particular circumstances and the severity of the condition being treated.

Determination of the appropriate, therapeutically-effective dose of regulatory T cells is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects. Preferably, a therapeutic composition that will be used is derived from the same species as the animal targeted for treatment, thereby minimizing a humoral response to the composition. The term “therapeutically effective” refers to an amount of cells that is sufficient to ameliorate, or in some manner reduce the symptoms associated with a disease or other undesired immune reaction. When used with reference to a method of this invention, the method is sufficiently effective to ameliorate, or in some manner reduce such symptoms.

In other embodiments of this invention, in which the methods are performed in vivo, compositions of matter are provided suitable for administration to patients in need of immunosuppression comprise a PLD inhibitor, optionally, a growth factor such as IL-2, and optionally, an activating antigen for which antigen-specific immunosuppression is desired.

Determination of the appropriate, therapeutically-effective dose of PLD inhibitor, activating antigen and growth factor is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects. The term “therapeutically effective” refers to an amount of PLD inhibitor, growth factor and activating antigen that is sufficient to ameliorate, or in some manner reduce the symptoms associated with a disease or other undesired immune reaction. When used with reference to a method of this invention, the method is sufficiently effective to ameliorate, or in some manner reduce such symptoms.

Compounds which are components of the compositions used in the methods of this invention can have prodrug forms. Any compound that will be converted in vivo to provide a biologically, pharmaceutically or therapeutically active form of a compound used in this invention is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

### EXAMPLES

The invention can be further understood by the following non-limiting examples.

The following examples show that a PLD-generated signal is required for expansion of effector T cells but is dispensable for proliferation of CD4⁺CD25⁺ regulatory T cells but is dispensable for expansion of CD4⁺CD25⁺ regulatory T cells. Inhibition of PLD-generated lipid signaling blocked proliferative responses by non-regulatory CD4⁺CD25⁻ T cells following TCR engagement. The same treatment had no significant effect on the proliferation of CD4⁺CD25⁺ T cells that developed regulatory functions under these conditions. The data identify a PLD-mediated signal as a key determinant of the outcome of T cell responses to TCR stimulation.

To study the role of PLD in primary murine T cells, we assessed the effect of 1-butanol treatment on splenic T cell proliferation. Carboxyfluorescein diacetate succinimidyl ester (CFSE)-labeled primary T cells were stimulated in vitro with anti-CD3 antibody in the presence of irradiated T cell-depleted antigen-presenting cells (APCs). This stimulus induced three to four rounds of cell division of both CD4 and CD8 T cells in 72 hours (FIG. 1A). Addition of 1-butanol to the culture medium abrogated cell division and the majority of T cells showed no division after 3 days. Tert-butanol (t-butanol), a tertiary alcohol which is not utilized by PLD in the transphosphatidylation reaction, had no significant effect on anti-CD3 induced cell division. Thus, modulation of PLD signal production with 1-butanol had a substantial anti-proliferative effect on T cells.

Materials and Methods

Mice, Antibodies, and Reagents

BALB/c, BM3 TCR and F1 (CBA×B6) mice were maintained in the specific pathogen-free facility at Medical College of Georgia. BM3TCR transgenic mice have been described previously (Auphan, N. et al. (1994), Eur. J. Immunol. 24:1572-1577). The Following antibodies were purchased from BD Biosciences Pharmingen (San Diego, Calif.): purified anti-CD3 (2C11), FITC-labeled anti-CD4 (RMA 4-5), -CD25 (7D4), -Thy1.2 (30-H12), PE-conjugated anti-CD4 (H129.19), -CD3 (145-2C11), -CD25 (PC61), biotinylated anti-CD4 (GK1.5), -CD8 (53-6.7), -CD25 (7D4,APC-labeled anti-CD4 (RMA4-5) and PerCP-conjugated anti-CD8 (53-6.7). 1-butanol and t-butanol were from Sigma (St. Louis, Mo.). Murine recombinant IL-2, IL-4 and IFN-γ were from Peprotech (Rocky Hill, N.J.). Anti-BM3 clonotypic (Ti-98) antibody has been reported previously (Buferne, M. et al. (1992), J. Immunol. 148:657-664). Cells were cultured in RPMI-1640 medium supplemented with 5% FCS, 50 μM 2-mercaptoethanol, 2 mM L-glutamine, 100 units/ml of penicillin and 100 μg/ml of streptomycin.

Cell Preparation

CD4⁺ T cell populations were prepared by eliminating B cells, adherent cells and CD8 T cells by panning using anti-CD8 and anti-mouse lg antibodies by a standard procedure (Coligan, J. E. (1999), Current Protocols in Immunology (John Wiley & Sons). CD4⁺CD25⁻ T cells were prepared by additional panning CD4⁺CD25⁻ with anti-CD25 antibody when CD4⁺CD25⁺ T cells were not required. For concurrent preparations of CD4⁺CD25⁺ and CD4⁺CD25⁻ T cells, cells were isolated by a MoFlo cell sorter (Dako Cytomation, Fort Collins, Colo.). Non-T cell populations were fractionated using a nylon-wool column for APC preparation as described (Julius, M. H. et al. (1973), Eur. J. Immunol. 3:645-649). Cell surface antigen analysis was performed by flow cytometry (FACS Calibur, Becton Dickinson, San Diego, Calif.).

Activation of T Cells, Assays for Cell Proliferation and Cytokine Production

For determination of cell division, T cells were labeled with 1 μM CFSE (Molecular Probes, Eugene, Oreg.) for 15 minutes at 37° C. CFSE-labeled T cells (5×10⁵ cells/ml) were cultured with 0.2 μg/ml of anti-CD3 with APCs (T cell-depleted splenocytes, γ-irradiated with 2000 rads, 8×10⁵ cells/ml). 1-butanol and t-butanol were added to a final concentration of 0.3%. After 72 hours, cells were harvested and stained with antibody against CD4 and CD8. The number of cell divisions was determined by flow cytometry. Exogenous IL-2 was added at 50 units/ml where indicated. For the proliferation assay, ³H-thymidine (0.5 μCi/well) was added for the last 6 hours of culture. Cytokine assays were performed by enzyme-linked immunoassays as previously described (Singh, N. et al. (1999), J. Immunol. 163:2373-2377).

Production and Functional Assays of CD4^{1-but}

For induction of CD4^{1-but}, CD4^{t-but} and CD4^{med} cells, CD4⁺ T cells were pre-incubated in medium containing 0.3% 1-butanol, 0.3% t-butanol or medium alone respectively (10⁶ cells/ml in 2 m!). After 15 hours, 1.5 ml of medium was replaced with 1.5 ml of medium containing 0.15 μg/ml of anti-CD3, γ-irradiated splenocytes (2000 rads, 5×10⁶ cells/well), 50 units/ml of recombinant murine IL-2, and 1-butanol or t-butanol (0.3% final concentration). On day 3, cells were harvested, washed and placed in medium containing 20 units/ml IL-2. On day 7, cells were washed, counted, and used for the regulatory function analysis. Numbers of cells obtained by this procedure is shown in Table 1.

TABLE 1
Numbers of live cells (CD4^{med}, CDR4^{t-but}, CD4^{1-but})
obtained from 1 × 10⁶ CD4⁺ T cells.
Results are shown as mean ⁺/− standard deviation
(×10⁶) from three independent experiments.

| Experiment | Medium | t-butanol | 1-butanol |
|---|---|---|---|
| 1 | 21 ± 3.6 | 13 ± 2 | 2.5 ± 0.5 |
| 2 | 19 ± 1 | 14 ± 2 | 3.4 ± 0.5 |
| 3 | 27 ± 1.4 | 18.5 ± 2.1 | 3.1 ± 0.3 |

In vitro Suppression Assay

To measure the suppressive activity of CD4^{1-but}, CD₄^{t-but}, and CD4^{med} cells, a range of doses of each population (6×10³˜5×10⁴ cells/well) were added to purified CD4⁺CD25⁻ T cells (2.5×10⁴ cells/well), which were stimulated by anti-CD3 antibody and γ-irradiated APC. Suppressive effects were measured by ³H-thymidine incorporation or by cytokine production.

Adoptive Transfer, in vivo Suppression Assay

F1 (CBA×B6) were injected intravenously with CD4^{med}, CD₄^{t-but}, CD4^{1-but} (4×10⁶ cells/mouse), or with PBS. Twenty-four hours later, mice were injected intravenously with BM3 TCR transgenic mouse splenocytes (5×10⁶ cells/mouse). Four days later, mice were sacrificed and spleens were fixed in 10% forrnaldehyde (Sigma, St. Louis, Mo.). 5 μμm sections were prepared from paraffin-embedded samples. Sections were stained either with hematoxylin and eosin or with Ti-98 (clonotypic antibody against BM3 TCR) using Dako^{r} ARK™ system and visualized according to the manufacturer's instructions. Quantitation of Ti-98 positive cells was performed over three sections using Bioquant Imaging Software.

RNA Interference, Transfection, RT .PCR

Expression constructs for siRNA of PLD were synthesized by Genscript Corporation (Scotch Plains, N.J.). A 21-nucleotide sequence (CCAACATMAGGTGATGCGAC [SEQ ID NO:1]) matching mouse pld2(1274-1294) and pld1(1328-1348, except for a substitution at position 18 from C to A) was used as the targeting sequence. Primary CD4⁺ T cells were transfected using 2 μμg siRNA construct and 200 ng plasmid encoding Renilla luciferase gene using an Amaxa electroporation system according to the conditions described previously (Lai, W. et al. (November, 2003), J. Immunol. Methods 282:93-102). Eighteen hours after transfection, 4×10⁴ live cells from each transfectant were used for functional assays. Total RNA was prepared using RNAwiz (Ambion, Austin, Tex.) according to the manufacturer's instructions. First-strand cDNA was prepared using Superscript II reverse transcriptase (Invitrogen, Carlsbad, Calif.). Polymerase chain reaction on cDNA was performed using Ex-Taq DNA polymerase (Takara, Otsu, Japan) for 35 cycles. The primers used for RT-PCR are:

| | |
|---|---|
| pld1; (⁺ strand) | |
| 5′-TGGCTGTCCCATAAMGCACMGT-3′, | [SEQ ID NO:2] |
| (− strand) | |
| 5′-TGGTATCCTGTGTCCCCCAGACCT-3′, | [SEQ ID NO:3] |
| pld2; (⁺ strand) | |
| 5′-GGTCCAAGAGGTGGCTGGT-3′, | [SEQ ID NO:4] |
| (− strand) | |
| 5′-CCGCCTTCCTCTTGAGCATAA 3′, | [SEQ ID NO:5] |
| g-3-pdh: (⁺ strand) | |
| 5′-CTCCCACTCTTCCACCTTCGA TGC-3′, | [SEQ ID NO:6] |
| (− strand) | |
| 5′-CCTCTCTTGCTCAGTGTCCTTGCT-3′, | [SEQ ID NO:7] |
| Foxp3: (⁺ strand) | |
| 5′-CCCAACCCTAGGCCAGCCAAG-3′, | [SEQ ID NO:8] |
| (− strand) | |
| 5′CACTTGCAGACTCCATTTGCCAG-3′. | [SEQ ID NO:9] |

To test the functional consequence on the reactivity of T cells activated in the presence of 1-butanol, we expanded CD4 T cells that were stimulated by anti-CD3 in the presence of 1-butanol (CD4^{1-but} cells), t-butanol (CD4^{t-but} cells), or medium alone (CD4^{med} cells) in culture medium free of alcohol in the presence of exogenous IL-2 (illustrated in FIG. 1G). Antigen reactivity of these T cells was tested by the proliferative response induced by anti-CD3 antibody. CD4^{t-but} cells and CD4^{med} cells responded equally well to stimulation (FIG. 1B). In contrast, CD4^{1-but} cells showed almost no response. Failure of CD4^{1-but} cells to respond to the secondary anti-CD3 stimulation was not due to loss of surface CD3 as shown by flow cytometric analyses (data not shown).

T cell unresponsiveness may be due to loss of antigen receptor reactivity (anergy) and/or the presence of regulatory T cells (Tregs) (Walker, L. S., and Abbas, A. K. (2002), Nat. Rev. Immunol. 2:11-19). To discriminate between these possibilities, CD4^{1-but} cells were tested for regulatory functions in secondary cultures. Freshly-isolated CD4⁺CD25⁻ T cells were stimulated by anti-CD3 antibody in coculture with irradiated T-depleted APCs. To this culture, either CD4^{1-but}, CD4^{t-but}, or CD4^{med} cells were added, and T cell proliferation was measured after 3 days. As shown in FIG. 1C, addition of CD4^{1-but} cells resulted in a strong inhibition of proliferation. The effect was evident even when CD₄^{1-but} cells were added to a four-fold excess of responder cells. Moreover, anti-CD3-induced production of IL-2 (FIG. 1D), IL4, and IFN-γ_{Y} (FIG. 1E) were all abrogated when CD4^{1-but} cells were added. Addition of CD4^{med} and CD₄^{t-but} cells had minimal effects on the proliferation of anti-CD3 stimulated CD4 T cells.

We next tested the suppressive activity of CD4^{1-but} cells in vivo using a well-characterized model of CD8 T cell mediated destruction of tissues (Mellor, A. L. et al. (Aug. 15, 2003), J. Immunol. 171:1652-1655), CD8 T cells from the BM3 transgenic mouse express a TCR that recognizes an allogenic epitope of H-2K^{b} (Reiser, J. B., et al (October 2000), Nat. Immunol. 291-297). When injected into mice of H-2^{bxk} haplotype, BM3-derived T cells expanded rapidly and caused tissue destruction as evidenced by loss of follicular structure in spleen ((Mellor, A. L. et al. (Aug. 15, 2003), J. Immunol. 171:1652-1655). To test the suppressive effects of CD4^{1-but} cells, H-2^{bxk} recipient mice were pre-treated with CD4^{med}, CD4^{t-but}, or CD4^{1-but} cells derived from H-2^{bxk} mice 24 hours prior to injection of BM3 T cells. Numbers of BM3-derived T cells per field of view were determined by anti-idiotype antibody staining. The results showed that BM3 T cells expanded significantly less in CD4^{1-but} cell-treated host mice than in CD4^{med} or CD4^{t-but} cell-treated host mice (FIGS. 1E and F). The regulatory function of CD4^{1-but} cells was further confirmed by the extent of tissue destruction (FIG. 1F, lower panels). Mice injected with CD4^{1-but} cells showed minimal signs of BM3-induced loss of the follicular architecture. In contrast; mice pretreated with CD₄^{med} or CD4^{t-but} cells showed tissue destruction similar to that observed with mice receiving no pretreatment. Together, these data confirmed that CD4^{1-but} cells have potent immunosuppressive activity that blocked aggressive T cell allo-responses in vivo.

Next; we addressed if the T cell regulatory function of CD4^{1-but} cells was due to differentiation of CD4⁺CD25⁻ T cells into regulatory T cells or a preferential expansion of CD4⁺CD25⁺ T cells. To discriminate between these possibilities, we removed CD25⁺ cells from CD4⁺ T cells and tested if anti-CD3 stimulation in the presence of 1-butanol induced regulatory function comparable to that observed with total CD4⁺ T cells. As shown in FIG. 2A, when CD4⁺CD25⁺ T cells were removed at the beginning of culture, 1-butanol and anti-CD3 treatment provoked no suppressive function in the expanded T cell population (FIG. 2A, left panel). This was in a stark contrast to the 1-butanol effect on total CD4⁺ T cells where strong suppressive activity was induced (FIG. 2A right panel). The data showed that T cell regulatory function of CD4^{1-but} requires the presence of CD4⁺CD25⁺ T cells at the beginning of culture. If differentiation is induced by 1-butanol, removal of CD4⁺CD25⁺ T cells from the initial isolate should not affect the induction of regulatory function. On the other hand, if regulatory T cells expand preferentially in the presence of 1-butanol, removal of CD4⁺CD25⁺ T cells would prevent their expansion and abolish the regulatory function. Thus, the data indicated that 1-butanol allowed preferential expansion of a pre-existing CD4⁺CD25⁺ T cell population. Indeed, removal of CD25⁺ T cells prior to 1-butanol treatment resulted in an 80% decrease in final yield (data not shown).

To examine directly the effect of modification of PLD signaling on T cell expansion, the rate of proliferation of CD4⁺ CD25⁻ and CD4+CD25⁺ cells in the presence of 1-butanol was quantified. As expected from a previous report, CD4⁺CD25⁺ cells did not respond to anti-CD3 stimulation and required exogenous IL-2 for proliferation (Takahashi, T. et al. (1998), Int. Immunol. 10:1969-80) (FIG. 2B). The presence of 1-butanol had no effect on the proliferation of CD4⁺CD25⁺ T cells following activation by anti-CD3 and IL-2. In contrast, 1-butanol substantially reduced the level of proliferation of CD4⁺CD25⁻ cells (80% reduction) whereas t-butanol caused no significant effect. The addition of exogenous IL-2 did not restore proliferation of 1-butanol treated CD4⁺ CD25⁻ cells (right panel). These results demonstrate that inhibition of PLD signaling blocked proliferation of CD4⁺CD25⁻ T cells but not CD4⁺CD25⁺ T cells, allowing preferential expansion of T cells with regulatory functions.

FoxP3 is an essential transcription factor for development and/or maintenance of regulatory T cells (Brunkow, M. E. et al. (2001), Nat. Genet. 27:68-73; Khattri, R. et al. (April, 2003), Nat. Immunol. 4:337-4342; Fontenot, J. D. et al. (April 2003), Nat. Immunol. 4:330-336; Hori, S. et al. (February 2003), Science 299:1057-1061) and is highly expressed in peripheral CD4⁺CD25⁺ T cells. CD4^{1-but} cells expressed Foxp3 mRNA at a significantly higher level than that found in CD4^{med} and CD₄^{t-but} cells (FIG. 2C, right panel). The levels of expression by CD4^{1-but} and purified CD4+CD25⁺ T cells were comparable (FIG. 2C, left panel). If CD4^{1-but} cells consist of T cells that are previously-defined regulatory T cells, they would be expected to express equivalent levels of FoxP3 to purified CD4⁺CD25⁺ T cells. This result confirms that 1-butanol treatment during CD4⁺ T cell activation enriched CD4⁺CD25⁺ regulatory T cells. All samples showed equivalent expression of pld1 and pld2 mRNA, the two major isoforms expressed in mammalian tissues (Exton, J. H. (2002), Rev Physiol. Biochem. Pharmacol. 144:1-94).

To elucidate mechanisms underlying the finding that PLD signaling is required for expansion of CD4⁺CD25⁻ effector T cells, but not for CD4⁺CD25⁺ regulatory T cells, we assessed the effects of 1-butanol treatment on expression of cytokines and cytokine receptors. 1-butanol treatment blocked production of IL-2 by CD4⁺CD25⁻ T cells (FIG. 3A). IFN-γ and IL-4 production was also abrogated by 1-butanol (FIG. 3). As reported previously (Takahashi, T. et aL (1998), Int. Immunol. 10:1969-1980), CD4⁺CD25⁺ T cells did not produce these cytokines after stimulation.

Next, we examined the effect of 1-butanol on high affinity IL-2 receptor (CD25) expression. Anti-CD3 stimulation induced CD25 in CD4⁺CD25⁻ T cells and upregulated the level of CD25 on CD4⁺CD25⁺ T cells (FIG. 3B). Up-regulation of CD25 expression in CD4⁺CD25⁺ T cells was not affected by treatment with 1-butanol. Additionally, 1-butanol did not impair the expression of CD69 by CD4⁺CD25⁺ T cells (not shown). In contrast, the presence of 1-butanol blocked the expression of CD25 by CD4⁺CD25⁻ T cells whereas t-butanol did not elicit a significant effect. Exogenous IL-2 did not restore CD25 expression (not shown). Since CD25 is a critical component of the high-affinity receptor for IL-2, inhibition of CD25 expression by 1-butanol would be expected to greatly impair T cell expansion of the CD4⁺CD25⁻ T cell population even in the presence of exogenous IL-2.

To further elucidate the role of PLD in T cell activation, TCR proximal signaling events were examined. First, the effect of 1-butanol on the elevation of intracellular Ca²⁺ was determined. When added to splenic CD4⁺ T cells, 1-butanol substantially impaired the anti-CD3-induced elevation of intracellular Ca²⁺ (dark line). Impairment was observed both in the initial and the later phases of activation. No significant effect was observed with t-butanol (thin line). Elevation of intracellular Ca²⁺ is required for activation of transcription factors, such as NF-AT, which are essential for CD25 and IL-2 expression (Crabtree, G. R. and Olson, E. N. (2002), Cell 109(Suppl):867-879; Hogan, P. G. et al. (Sep. 15, 200e), Genes Dev. 17:2205-2032). Thus, inhibition of Ca²⁺ elevation by 1-butanol may be a cause of impaired IL-2 production and CD25 expression by CD4⁺ CD25⁻ T cells.

TCR stimulation also induces activation of the Ras/ERK pathway, and sustained ERK activation is essential for IL-2 production (Iwashima, M. (May, 2003), Immunol. Review 192; T. Koike et al., J. Biol. Chem. 278:15685-15692). The role of PLD in ERK activation was examined by intracellular staining with antibodies that recognize the phosphorylated (active) form of ERK. Anti-CD3 stimulation induced ERK phosphorylation in CD4 T cells (FIG. 3D). The presence of 1-butanol abolished this CD3-induced elevation of phosphorylated ERK whereas t-butanol had no detectable effect. Together, these data indicate that PLD activity is essential for early signaling events that are required for both Ca²⁺ elevation and ERK activation.

Although the effect of 1-butanol as a modulator of PLD signaling is well established (Liscovitch, M. et al. (2002), Biochem. J. 345(Pt 3):401-415), we examined its specificity using short interfering RNA (siRNA)-mediated gene knock-down of PLD. Splenic CD4 T cells were transfected by electroporation (Lai, W. et al. (November 2003), J. Immunol. Methods 282:93-102) with an expression construct for siRNA targeted toward both PLD1 and PLD2. The effect of the siRNA construct in reducing PLD1 and PLD2 levels was confirmed by RT-PCR (FIG. 4A). When stimulated with anti-CD3 antibody, cells transfected with the construct for PLD siRNA showed a significant reduction in proliferation and IL-2 production (FIG. 4B). Levels of IL-2 production and proliferation of CD4 T cells transfected with the control siRNA construct were equivalent to those of mock-transfected T cells. Thus, the level of PLD expression affects T cell responsiveness. These data support the conclusion that the effect of 1-butanol treatment on CD4 T cell proliferation is caused by loss of PLD-generated signals.

The data presented here demonstrate that PLD plays an essential role in the expansion of CD4⁺CD25⁻ T cells following activation. However, this requirement is selective since inhibition of PLD signaling had no effect on expansion of CD4⁺CD25⁺ regulatory T cells. Inhibition of PLD function impaired TCR proximal signaling events (Ca²⁺ and ERK activation) and blocked induction of cytokines and surface antigen expression in CD4⁺CD25⁻ T cells. On the other hand, up-regulation of CD25 expression by CD4⁺CD25⁺ T cells was independent of PLD signaling. Recently, it was shown that Raf and Kinase Suppressor of Ras (KSR) bind PA, the product of PLD-mediated phospholipid hydrolysis (Andresen, B. T. et al. (2002) FEBS Letts 531:65-68). Since both Raf and KSR play critical roles in the regulation of Ras-induced ERK activation, PLD may be required to induce maximal Raf and thus ERK activation. Indeed, it was reported that AP-1, a transcription factor that is activated by the ERK pathway, was regulated in a PLD dependent manner in Jurkat cells (Mollinedo, F. et al. (1994), J. Immunol. 153:2457-2469). It should be also noted that PLD has been shown to couple the high-affinity receptor for IgG (FcyRI) to the release of intracellular Ca²⁺ (Melendez, A. et al. (1998), Curr. Biol. 8: 210-221; Melendez, A. J., et al. (2001), Blood 98:3421-3428).

Differences in PLD signaling requirements of CD4⁺CD25⁻ and CD4⁺CD25⁺ T cells indicate that PLD plays distinct roles for activation in CD4⁺CD25⁻ versus CD4⁺CD25⁺T cells. Levels of PLD mRNA expression are comparable between CD4⁺CD25⁻ and CD4⁺CD25⁺ T cells (FIG. 2C). Our data shows that anti-CD3 stimulation upregulates PLD activity in CD4⁺CD25⁻ T cells approximately by two-fold as shown previously for human and murine T cells (Stewart, S. J. et al. (1991), Cell Regul. 2:841-850; Reid, P. A. et al. (1997), Immunology 90:250-256) In contrast, PLD activity in CD4⁺CD25⁺ T cells is constitutively high both in their resting and activated stages. Thus, PLD may be regulated differently in CD4⁺CD25⁻ and CD4⁺CD25⁺ T cells.

The results provide a simple method for expansion of regulatory T cells without the addition of various growth factors as reported previously (Wahl, S. M. and Chen, W. (2003), Immunol. Res. 28:167-179; Horwitz, D. A. et al. (October 2003), J. Leukoc. Biol. 74:471-478). The efficiency of this procedure will facilitate therapeutic treatments for autoimmunity, allergy, and tissue transplantation.

### Example of the Use of Adenosine as a PLD Inhibitor

Since adenosine has been described to inhibit PLD activation in neutrophils (Thibault, N., et al. (2000), Blood (95(2):519-527; Grenier, S. et al., J. Leukoc. Biol. 73(4):530-539), the effect of adenosine on T cell antigen receptor (TCR)-induced PLD activity was tested. As shown in FIG. 5, treatment of primary T cells with adenosine completely abrogated PLD activity induced by TCR stimulation. The data shows that adenosine and its derivatives that act as agonists for adenosine receptors function as effective inhibitors of TCR-induced PLD activation in place of primary alcohol, and are useful for patient treatment in accordance with the methods of this invention.

Mouse CD4 T cells were labeled with ³H-oleate to measure PLD activity (Zheng, et al. (2003), Biochim. Biophys. Acta. 1643(1-3):25-36). Cells were then washed and activated by plate-bound anti-CD3 for 40 minutes in the presence of 0.5% of ethanol (open bar). Effect of adenosine was examined using the medium containing 100 μM of adenosine (closed bar). Cells were harvested and lipid extracts of cells were separated on TLC plates and bands corresponding to phosphatidic acid (PA) (FIG. 5A) and phosphatidylethanol (Pet) (FIG. 5B) were excised and counted by liquid scintillation.

### Example of the Use of siRNA to Block PLD Function in T Cell Expansion

Since a clear difference was observed in the level of PLD expression between CD4⁺CD25⁻ and CD4⁺CD25⁺ cells, the functional relevance of PLD in T cell activation was tested. To this end, siRNA-based gene knockdown of PLD in primary T cells was employed. When splenic CD4 T cells were transfected with an siRNA expression construct for PLD1/2 (siPLD), both mRNA and protein levels of PLD1/2 were reduced significantly (FIG. 5A). When stimulated with anti-D3 antibodies, siRNA-transfected CD4 cells showed more than 50% reduction in IL-2 production and proliferation (FIG. 6B). To test whether PLD gene knockdown affected expansion of CD4⁺CD25⁻ and CD4⁺CD25⁺ T cells equally, the levels of Foxp3 m RNA that was preferentially expressed by CD4⁺CD25⁺T cells was examined. Real time PCT-based quantitation of Foxp3 mRNA showed approximately a 300% increase in siPLD-transfected cells against control (FIG. 6C). The data indicated that PLD plays a critical role in TCR-induced expansion of CD4⁺CD25⁻, but not CD4⁺CD25⁺ T cells. Moreover, this data shows that gene knockdown of PLD is also an effective procedure to block PLD function and enrich regulatory T cells in place or in combination with primary alcohol.

Purified CD4T cells were transfected with the expression cassette targeted toward both PLD1 and 2 (1 nucleotide difference). As a control, cells transfected with the expression cassette for EGFP (U6-EGFP) or with no DNA were examined. Eighteen hours after transfection, total levels were determined for PLD1, PLD2 and G3PDH by RT-PCR. Protein levels were determined by Western blot with anti-PLD1 (FIG. 6A, top), PLD2 (middle), and Lck (bottom) antibodies.

The effect of PLD siRNA on anti-CD3-induced T cell proliferation and IL-2 product was examined. See FIG. 6B. CD4 T cells transfected as described above were stimulated with anti-CD3 and APCs. Proliferation (after 72 hours) and IL-2 production (after 24 hours) were analyzed for each sample.

FIG. 6C shows Foxp3 expression by cells treated with siRNA for PLD. Cells were transfected and stimulated as described above. mRNA was isolated three days after stimulation and FoxP3 mRNA level was determined by real time PCR. The results from two independent experiments are shown as the relative mRNA levels of FoxP3 against G3PDH.

### Example of Use of Autologous Regulatory T Cells for Application in Autoimmune Disorders

In a patient in need of treatment for an autoimmune disorder, regulatory T cells from the patient are selectively isolated or expanded. For instance, a patient with systemic lupus erythematosus, arthritis, or other disorder. A population of T cells obtained from the patient. These T cells are exposed in culture to a primary alcohol and anti-CD3 antibody or specific antigens. After a period of time, the effector T cells are eliminated. The population is optionally treated with a T cell growth factor such as IL-2. The regulatory T cells in the population are thus selected or expanded in comparison to effector T cells. The processed regulatory T cells are then optionally further purified and administered to the patient. The processed regulatory T cells in the patient are now able to suppress effector T cell responses. Such suppression can alleviate clinical symptoms or progression of the autoimmune disorder.

### Example of Use of Autologous Regulatory T Cells for Application in Transplant Procedures

In a patient in need of a transplanted cell, tissue, or organ from a heterologous donor, regulatory T cells from the patient are selectively isolated or expanded. For instance, a sibling or unrelated person serves as a transplant donation source. The source material is characterized such as by tissue typing. A sample from the donation source or other material defined as comprising an antigenic composition similar to that of the donation source is used to contact ex vivo a population of T cells obtained from the patient. The T cells are also exposed in culture to a primary alcohol. After a period of time, the effector T cells are eliminated. The population is optionally treated with a T cell growth factor such as IL-2. The regulatory T cells in the population are thus selected or expanded in comparison to effector T cells. The processed regulatory T cells are then optionally further purified and administered to the patient. The regulatory T cells in the transplant recipient are able to suppress effector T cell responses to the incoming transplant material. The procedure can optionally be performed before or after the transplant. Preferably the autologous regulatory T cells are processed and administered in advance of the transplant.

### Example of Vaccine Applications

In a classic sense, a vaccine is used to provoke a positive response against an undesirable antigen source such as pathogenic viruses or bacteria. Here, however, a vaccine is developed to selectively enhance the ability of regulatory T cells to achieve a down regulation of an immune response. For instance, a vaccine is prepared for a disorder such as Type I diabetes or a food allergy. As described herein, a T cell population is obtained from a patient. Ex vivo, the T cells are contacted with a primary alcohol. They can also be contacted with an antigen relevant to the condition, for example a pancreatic islet cell antigen for diabetes, a food allergen, or a DNA molecule for lupus. The T cells are further optionally contacted with a cytokine such as T cell growth factor. After a period of time, the effector T cells are at least partially eliminated. The regulatory T cells in the population are thus selected or expanded in comparison to effector T cells. After optional further purification, the processed regulatory T cells are then administered to the patient. The regulatory T cells in the patient are now able to suppress effector T cell responses to the offending antigen.

In a specific allergic condition, the allergen is a pollen. The vaccine is prepared as a composition of a PLD inhibitor in an eye drop formulation. In another condition, the allergen is a skin allergen. A composition is a PLD inhibitor in a skin cream formulation or treated transdermal patch optionally with an antigen. For a food allergen, a composition is a PLD inhibitor with an antigenic solution or solid bolus for oral ingestion. For an upper respiratory or systemic antigen, a composition is a PLD inhibitor in an inhalable formulation, optionally with an appropriately formulated antigen solution or antigen particle composition.

### Example of Expansion of CD4 Foxp3⁺ Cells in the Presence of 1-butanol

We expanded CD4 T cells by stimulating with anti-CD3 in the presence of 1-butanol (CD4^{1-but} cells), t-butanol (CD4^{t-but} cells), or medium alone (CD4^{med} cells) (illustrated in FIG. 7A). After 3 days of culture with alcohol, cells were expanded in the medium free of alcohol but containing IL-2 for 4 days and analyzed by flow cytometry with Foxp3 and CTLA-4. As shown in FIG. 7B, CD4^{1-but} cells contain over 80% of cells expressing Foxp3 and CTLA-4. In contrast, we obtained almost no cells expressing Foxp3 and CTLA-4 from culture with t-butanol or medium alone. Total cell number of Foxp3⁺ cells show that these cells expanded more than 10-fold in one week in the presence of 1-butanol, but not in controls (FIG. 7C).

### Example of Preferential Expansion of CD4⁺CD25⁺ Cells in the Presence of 1-butanol

We also examined the effect of 1-alcohol on the proliferation of CD4⁺CD25⁻ and CD4⁺CD25⁺ cells. CD4⁺CD25⁻ and CD4⁺CD25⁺ cells were isolated from splenocytes using a MoFlo cell sorter. Each cell type was stimulated with anti-CD3 antibody in the presence of irradiated APCs with or without the addition of exogenous IL-2. Proliferation was measured by ³H-thymidine incorporation on day 3. As shown in FIG. 7D, CD4⁺CD25⁻ T cells respond vigorously to stimulation either in the absence of (left panel) or presence of (right panel) exogenous IL-2. CD4⁺CD25⁺ cells did not respond to anti-CD3 stimulation and required exogenous IL-2 for proliferation. 1-butanol substantially reduced the level of proliferation of CD4⁺CD25⁻ cells (80% reduction) whereas t-butanol had no significant effect. In contrast, the presence of 1-butanol had no effect on the proliferation of CD4⁺CD25⁺ T cells following activation with anti-CD3 and IL-2. The addition of exogenous IL-2 did not rescue the proliferation of 1-butanol treated CD4⁺CD25⁻ cells (right panel). These results demonstrate that treatment with 1-butanol blocked proliferation of CD4⁺CD25⁻ T cells but not CD4⁺CD25⁺ T cells, allowing preferential expansion of CD4⁺CD25⁺ T cells expressing Foxp3.

### Example of Plate-bound Antibody-based Stimulation of CD4⁺CD25⁺ T Cells

To expand regulatory T cells (Tregs) without contamination from antigen-presenting cells, we developed a novel procedure using plate-bound antibody-based stimulation of Tregs. CD4⁺CD25⁺ T cells were sorted by MoFlo and rested overnight in complete medium at 4° C. Polystyrene uncoated/untreated plates were coated with 5 μg/ml of anti-CD3 (ebioscience, clone 145-2C11) plus 5 μg/ml of anti-CD28 (ebioscience) overnight at room temperature in borate buffer (0.1M pH 8.5, 2 ml/plate). The next day, the plate was blocked with 1% fatty acid-free bovine serum albumin (BSA) in borate buffer (0.1M pH 8.5) for 60 minutes. Plates were washed with phosphate buffered saline (PBS) twice and 0.5×10⁶ cells were placed per plate in 5 ml medium containing 10 ng/ml of IL-2. Four days later, cells were split 1:4 on newly-coated plates. The cell density was monitored after day 6 to keep the density under 2×10⁶/ml. About 100-200-fold expansion of Tregs was observed on days 7-8. Expanded Tregs showed regulatory functions as freshly-isolated Tregs. Fold expansion of Tregs by this procedure from four independent experiments is are shown in FIG. 8.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof.

The invention has been described with reference to various specific and preferred embodiments and techniques. However, it should be understood that many variations and modifications can be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example or illustration and not of limitation.

1. A method for selectively increasing proliferation of regulatory T cells compared to effector T cells comprising:
a) contacting a T cell population, wherein the population comprises regulatory T cells and optionally effector T cells, with a phospholipase D (PLD) inhibitor in an amount effective to selectively inhibit said effector T cells;
b) activating said regulatory T cells, and effector T cells if present; and
c) allowing proliferation of said regulatory T cells, and elimination of said effector T cells if present.

2. The method of claim 1 also comprising contacting said T cell population with a growth factor in an amount sufficient to promote proliferation of said regulatory T cells.

3. The method of claim 1 wherein said phospholipase D inhibitor is a compound comprising at least one primary hydroxyl group or sulfhydryl group conjugated to a physiologically acceptable moiety through a linear spacer group n carbon atoms or n heteroatoms in length, wherein n is an integer from 3 to 20.

4. The method of claim 1 wherein said phospholipase D inhibitor is a primary alcohol.

5. The method of claim 3 wherein said phospholipase D inhibitor is 1-butanol or 1-propanol.

6. The method of claim 1 wherein said phospholipase D inhibitor is a serine protease inhibitor.

7. The method of claim 1 wherein said phospholipase D inhibitor is adenosine or an adenosine derivative.

8. The method of claim 1 wherein said phospholipase D inhibitor is a PLD1 inhibitor.

9. The method of claim 1 wherein said method is performed in vitro.

10. The method of claim 9 wherein the proliferated regulatory T cells are administered to a patient in need of immunosuppression.

11. The method of claim 9 wherein the regulatory T cells are CD4⁺CD25⁺ cells.

12. The method of claim 9 wherein said method is performed in vivo.

13. The method of claim 1 wherein the regulatory T cells have been activated with anti-CD3 antibody.

14. The method of claim 1 in which said proliferation produces regulatory T cells capable of suppressing activity of helper T cells to a specific antigen, wherein said regulatory T cells have been activated in the presence of said specific antigen.

15. The method of claim 14 wherein said regulatory T cells are proliferated to a clinically relevant number.

16. The method of claim 14 performed in vivo by administering to a patient in need of antigen-specific immunosuppression, a specific antigen to which antigen-specific immunosuppression is needed, and a PLD inhibitor.

17. The method of claim 13 wherein said administering is done via a vehicle selected from the group consisting of inhalation sprays, eye drops, intravenous injection carriers, oral delivery carriers, and topical delivery carriers.

18. The method of claim 17 also comprising administering a growth stimulator.

19. The method of claim 18 wherein said growth stimulator is IL-2.

20. A method for suppressing an immune reaction in a patient in need of immunosuppression comprising administering to said patient:
(a) a phospholipase D inhibitor comprising a primary alcohol selected from the group consisting of 1-propanol, 1-butanol, and ethanol in an amount effective to selectively produce a T cell population enriched in regulatory CD4⁺CD25⁺ T cells in said patient capable of suppressing an immune response of effector T cells in said patient by a measurable amount;
(b) an antigen in an amount effective to activate said regulatory or effector T cells;
(c) optionally, a growth factor in an amount effective to stimulate selection or expansion of said antigen-specific regulatory T cells.

21. The method of claim 20 wherein said antigen is a selected antigen to which specific immunosuppression in said patient is desired.

22. The method of claim 20 wherein said growth factor is IL-2.

23. The method of claim 20 wherein said patient in need of immunosuppression is a patient at risk for developing a condition selected from the group consisting of: rheumatoid arthritis, lupus, multiple sclerosis, inflammatory bowel disease, insulin-dependent diabetes mellitus, autoimmune thyroid disease, anti-tubular basement membrane disease (kidney), Sjogren's syndrome, ankylosing spondylitis, ureoetinitis, allograft rejection, transplant rejection, food allergies, non-food allergies, stroke, infection-induced tissue destruction by immune responses, and asthma.

24. A method of autologous cell therapy for effecting antigen-specific immunosuppression comprising:
(a) collecting T cells from a patient;
(b) activating said T cells by contacting them with an antigen;
(c) culturing said T cells ex vivo in the presence of a primary alcohol selected from the group consisting of 1-butanol or 1-propanol and a growth factor selected from the group consisting of IL-2 and TGF-β, IL-7, IL-12, and IL-10 in an effective amount to promote selection or expansion of regulatory T cells in culture;
(d) expanding the regulatory T cells in said culture until a clinically significant number of regulatory T cells capable of specifically suppressing immune response to the selected antigen has been produced; and
(e) administering said regulatory T cells to a patient in need of said antigen-specific immunosuppression.

25. A pharmaceutical composition for treatment of a patient in need of antigen-specific immunosuppression comprising:
(a) a phospholipase D inhibitor; and
(b) an antigen for which said antigen-specific immunosuppression is desired.

26. The composition of claim 25 also comprising a T cell growth stimulator.

27. The composition of claim 25 wherein said phospholipase D inhibitor is 1-butanol or 1-propanol.

28. The composition of claim 25 wherein said T cell growth stimulator is IL-2.

29. The composition of claim 25 which is a vaccine.

30. The composition of claim 25 which also comprises a suitable carrier for a mode of administration selected from the group consisting of topical administration, nasal infusion, inhalation, delivery to the eyes, subcutaneous injection, intravenous injection, intramuscular injection, implantation of pellets, and oral ingestion.

31. A pharmaceutical composition of matter suitable for administration to patients in need of immunosuppression comprising a clinically relevant number of regulatory T cells in a suitable pharmaceutical carrier.

32. The composition of claim 31 also comprising an antigen in an amount effective to activate T cells.

33. The composition of claim 31 wherein said pharmaceutical carrier is a carrier suitable for administration via injection or orally.
